(19) United States
(12) Patent Application Publication
(10) Pub. No.: US 2007/0003926 A1
(43) Pub. Date: Jan. 4, 2007

(54) Nucleic acid sequences from drosophila melanogaster that encode proteins essential for viability and uses thereof

(76) Inventors: Lynn Stam, Raleigh, NC (US); Kim Kamdar, LaJolla, CA (US); Eric Spana, Raleigh, NC (US); Jane Bachmann, Basel (CH)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 10/527,571

(22) PCT Filed: Aug. 8, 2003
(86) PCT No.: PCT/US03/24982

§ 371 Date: Mar. 14, 2005

Related U.S. Application Data
(60) Provisional application No. 60/422,377, filed on Oct. 30, 2002.

(51) Int. Cl. C12Q 1/68; G01N 33/53; A01N 61/00

(52) U.S. Cl. 435/6; 435/7.1; 514/1

(57) ABSTRACT

Nucleotide sequences are isolated from Drosophila melanogaster that code for proteins essential for viability. These proteins are useful for discovering new insecticides based on the essentiality of the nucleotide sequences for Drosophila viability. Further provided are recombinant proteins and methods for identifying inhibitors to these proteins. Protein inhibitors active in the methods disclosed herein are useful as insecticidal, ectoparasiticidal, antiparasitic, anthementhic and acaracidal agents.

This application claims the benefit of U.S. Provisional Patent Application Ser. No. 60/422,377 filed Oct. 30, 2002, which is incorporated by reference in its entirety.

The Sequence Listing associated with the instant disclosure has been submitted as a 2.62 megabyte file on CD-R (in duplicate) instead of on paper. Each CD-R is marked in indelible ink to identify the Applicants, Title, File Name (70131WOPCT.ST25.txt), Creation Date (Aug. 7, 2003), Computer System (IBM-PC/MS-DOS/MS-Windows), and Docket No. (70131 WOPCT). The Sequence Listing submitted on CD-R is hereby incorporated by reference into the instant disclosure.

### FIELD OF INVENTION

The present invention pertains to nucleic acid sequences isolated from Drosophila melanogaster that encode proteins essential for viability. The invention particularly relates to methods of using these proteins as insecticide targets, based on this essentiality.

### BACKGROUND OF THE INVENTION

Insects contribute or cause many human and animal diseases, and are responsible for substantial agricultural and property damage. The societal costs associated with insect pests in dollars, time and suffering are monumental. The total worldwide market size for insecticide crop protection is over $5 billion. To combat these problems, insecticidal compounds have been developed and employed.

The idea to use chemicals for insect control is not new. The scientific use of pesticides started with the introduction of arsenical insecticides and organic compounds such as tar, petroleum oils, and dinitrophenol emulsions at the end of the last century. But, the systematic search for synthetic organic insecticides was only launched after the discovery of the insecticidal properties of DDT in 1939. After World War II, chemical research concentrated mainly on chlorinated hydrocarbons and cyclodienes, which all require high rates of application and have a rather broad spectrum of activity. Most of them are persistent in the environment and may pose a significant risk for accumulation in the food chain. Today the use of these chemicals is very much restricted.

From this point, the major emphasis in research has been given to organophosphates and carbamates, which are readily degradable in the environment with little tendency for bioaccumulation. The toxicity of these compounds varies within a broad range from medium to highly toxic. Organophosphates and carbamates are still widely use, although the more toxic ones are banned in certain countries. The formamidines have as their major advantage a different mode of action and their selectivity, which made them suitable for use in IPM (insect pest management) programs. They are easily degradable with no accumulation potential, but for toxicological reasons some have had to be withdrawn from the market.

For the past decade, insecticide research has concentrated on leadfinding for new chemical structures interfering with new target mechanisms. The chances for success are rather remote, because the hurdles for the registration of a new insecticide are set very high. Toxicological aspects, insecticide resistance, environmental behavior, and IPM fitness are some of the critical factors that have to be considered together with economical factors.

Novel insecticides can now be discovered using high-throughput screens that implement recombinant DNA technology. Proteins found to be essential to insect viability can be recombinantly produced through standard molecular biological techniques and utilized as insecticide targets in screens for novel inhibitors of the enzymes' activity. The novel inhibitors discovered through such screens may then be used as insecticides to control undesirable insect infestation.

However, as the world population continues to grow, there will be increasing food shortages. Therefore, there exists continuing need to find new, effective and economic insecticides.

### SUMMARY OF THE INVENTION

In view of these needs, it is one object of the invention to provide essential genes in insects such as Drosophila melanogaster. It is another object to provide the essential proteins encoded by these essential genes for assay development to identity inhibitory compounds with insecticidal activity. It is still another object of the present invention to provide an effective and beneficial method for identifying new or improved insecticides using the essential proteins of the invention.

In furtherance of these and other objects, the present invention provides DNA molecules comprising nucleotide sequences isolated from Drosophila melanogaster that encode proteins essential for viability. The inventors are the first to demonstrate that the nucleotide sequences of the invention are essential for viability. This knowledge is exploited to provide novel insecticide modes of action. One advantage of the present invention is that the proteins encoded by the essential nucleotide sequences provide the bases for assays designed to easily and rapidly identify novel insecticides.

Disruption of the nucleotide sequences or messenger RNA of the invention demonstrates that the activity of each corresponding encoded protein is essential for Drosophila viability. Genetic results show that when each nucleotide sequence of the invention is mutated in Drosophila or disrupted at the transcription level, the resulting phenotype is lethal. This demonstrates a critical role for the protein encoded by the mutated nucleotide sequence. This further implies that chemicals that inhibit the expression of the protein when in contact with insects are likely to have detrimental effects on insects and are potentially good insecticide candidates. The present invention therefore provides methods of using the disclosed nucleotide sequences or proteins encoded thereby to identify inhibitors thereof. The inhibitors can then be used as insecticides to kill undesirable insect populations where crops are grown, particularly agronomically important crops such as maize, and other cereal crops such as wheat, oats, rye, sorgum, rice, barley, millet, turf and forage grasses and the like, as well as cotton, sugar cane, sugar beet, oilseed rape, soybeans, vegetable crops and fruits.

The present invention accordingly provides cDNA sequences derived from Drosophila melanogaster. In one embodiment, the present invention provides an isolated DNA molecule comprising a nucleotide sequence selected from the group consisting of the even numbered SEQ ID NOs:14-380. In another embodiment, the present invention provides an isolated DNA molecule comprising a nucleotide sequence that encodes a protein selected from the group consisting of the odd numbered SEQ ID NOs:15-381.

The present invention also provides a chimeric construct comprising a promoter operatively linked to a DNA molecule according to the present invention, wherein the promoter is preferably functional in a eukaryote, wherein the promoter is preferably heterologous to the DNA molecule. The present invention further provides a recombinant vector comprising a chimeric construct according to the present invention, wherein said vector is capable of being stably transformed into a host cell. The present invention still further provides a host cell comprising a DNA molecule according to the present invention, wherein said DNA molecule is preferably expressible in the cell. The host cell is preferably selected from the group consisting of an insect cell, a yeast cell, and a prokaryotic cell.

The present invention also provides proteins essential for Drosophila melanogaster viability. In one embodiment, the present invention provides an isolated protein comprising an amino acid sequence selected from the group consisting of the odd numbered SEQ ID NOs:15-361. In accordance with another embodiment, the present invention also relates to the recombinant production of proteins of the invention and methods of using the proteins of the invention in assays for identifying compounds that interact with the protein.

In another preferred embodiment, the present invention describes a method for identifying chemicals having the ability to inhibit the activity of the disclosed proteins. In a preferred embodiment, the present invention provides a method for selecting compounds that interact with a protein of the invention, comprising: (a) expressing a DNA molecule according to the present invention to generate the corresponding protein of the invention, (b) testing a compound suspected of having the ability to interact with the protein expressed in step (a), and (c) selecting compounds that interact with the protein in step (b).

Other objects and advantages of the present invention will become apparent to those skilled in the art and from a study of the following description of the invention and non-limiting examples. The entire contents of all publications mentioned herein are hereby incorporated by reference.

### BRIEF DESCRIPTION OF THE SEQUENCES IN THE SEQUENCE LISTING

SEQ ID NOs:1-13 are PCR primers.

Even numbered SEQ ID NOs:14-380 are nucleotide sequences described in the table below.

Odd numbered SEQ ID NOs:15-381 are protein sequences encoded by the immediately preceding nucleotide sequence, e.g., SEQ ID NO:15 is the protein encoded by the nucleotide sequence of SEQ ID NO:14, SEQ ID NO:17 is the protein encoded by the nucleotide sequence of SEQ ID NO:16, etc.

TABLE 1
Drosophila Sequences

| seq | Inventor's | | | | |
|---|---|---|---|---|---|
| ID | reference | function | Domains | Best blast hit | score |
| 14-15 | CT28483 | CG10260 | PI3Ka, PI3_4_KINASE_1, | (D83538) 230 kDa | 1600 |
| | | EG: BACR7C10.2 protein | PI3_4_KINASE_2, | phosphatidylinositol 4-kinase | |
| | | kinase, 1- | PI3_4_KINASE_3, | [Rattus norvegicus] | |
| | | phosphatidylinositol 4- | PI3_PI4_kinase | | |
| | | kinase | | | |
| 16-17 | CT28925 | CG10365 unknown | | hypothetical protein MGC4504 | 185 |
| | | | | [Homo sapiens] | |
| 18-19 | CT29122 | CG10370 Tbp-1 Tat- | AAA, ATP_GTP_A, | Q63569\|PRSA_RAT 26S | 720 |
| | | binding protein-1, | MITOCH_CARRIER | PROTEASE REGULATORY | |
| | | Proteasome 26S | | SUBUNIT 6A (TAT-BINDING | |
| | | regulatory subunit 6A, | | PROTEIN 1) (TBP-1) | |
| | | multicatalytic | | | |
| | | endopeptidase, | | | |
| 20-21 | CT29492 | CG10545 Gb13F G | GPROTEINB, | GBB1_CAEEL GUANINE | 619 |
| | | protein b-subunit 13F, G- | GPROTEINBRPT, WD40, | NUCLEOTIDE-BINDING | |
| | | protein coupled receptor, | WD40_REGION, | PROTEIN BETA SUBUNIT 1 | |
| | | protein signaling pathway | WD_REPEATS | | |
| 22-23 | CT30008 | CG10701 Moe Dmoesin, | BAND41, BAND_41_1, | Homo sapiens ‘moesin’ | |
| | | motor involved in | BAND_41_2, BAND_41_3, | gi: 4505257 | |
| | | cytoskeleton organization | Band_41, ERM, ERMFAMILY | | |
| | | and biogenesis | | | |
| 24-25 | CT30208 | CG10776 wit | PROTEIN_KINASE_ATP, | NP_031587.1\| (NM_007561) | 362 |
| | | Serine/threonine kinase- | PROTEIN_KINASE_DOM, | bone morphogenic protein | |
| | | D; wishful thinking, a type | TGFB_RECEPTOR, pkinase | receptor, type II | |
| | | II transforming growth | | | |
| | | factor beta receptor | | | |
| | | involved in protein | | | |
| | | phosphorylation | | | |
| 26-27 | CT30807 | CG10997 chloride | | NP_001280.2\| (NM_001289) | 119 |
| | | channel? | | chloride intracellular channel 2 | |
| | | | | [Homo sapiens] | |
| 28-29 | CT30887 | CG11033 unknown | | NP_036440.1\| (NM_012308) F- | 431 |
| | | | | box and leucine-rich repeat | |
| | | | | protein 11 | |
| 30-31 | CT31117 | CG11130 Rtc1 RNA 3′ | | Q9Y2P8\|RCL1_HUMAN RNA | 326 |
| | | terminal phosphate | | 3′-TERMINAL PHOSPHATE | |
| | | cyclase, Rtc1 | | CYCLASE-LIKE PROTEIN | |
| | | | | (HSPC338) | |
| 32-33 | CT1249 | CG1114 Weak similarity | | NP_071334.1\| (NM_022051) | 249 |
| | | with apoptosis protein RP- | | egl nine homolog 1 (C. elegans) | |
| | | 8, | | | |
| 34-35 | CT1483 | CG1119 Gnf1 Germ line | ATP_GTP_A, BRCT, | A49651 replication factor C | 661 |
| | | transcription factor 1, | BRCT_DOMAIN, NLS_BP, | large subunit - human | |
| | | DNA binding/DNA | RFC | | |
| | | replication factor | | | |
| 36-37 | CT7860 | CG11190 unknown | | BAB60854.1\| (AB057724) | 387 |
| | | | | phosphatidyl inositol glycan | |
| | | | | class T [Homo sapiens] | |
| 38-39 | CT1834 | CG1135 unknown | FHA, FHA_DOMAIN | NP_006328.1\| (NM_006337) | 383 |
| | | | | microspherule protein 1; cell | |
| | | | | cycle-regulated factor | |
| 40-41 | CT31875 | CG11418 EG: 8D8.8 | | NP_060579.1\| (NM_018109) | 252 |
| | | involved in cell cycle | | hypothetical protein FLJ10486 | |
| | | | | [Homo sapiens] | |
| 42-43 | CT36241 | CG11452 unknown | | none | |
| 44-45 | CT1993 | CG1149 MstProx | LRRNT | Homo sapiens ‘toll-like | |
| | | MstProx, transmembrane | | receptor1’ gi: 4507527 | |
| | | receptor involved in | | | |
| | | defense response | | | |
| 46-47 | CT34608 | CG11511 similarity to | ZINC_FINGER_C2H2, | AAC78286.1\| (AF032674) | 128 |
| | | broad-complex Z2- | ZINC_FINGER_C2H2_2, zf- | broad-complex Z2-isoform | |
| | | isoform | C2H2 | [Manduca sexta] | |
| 48-49 | CT5404 | CG11595 unknown | | none | |
| 50-51 | CT17728 | CG11779 receptor - | | XP_049282.1\| (XM_049282) | 436 |
| | | mitochondrial | | translocase of inner | |
| | | transporter??? | | mitochondrial membrane 44 | |
| | | | | homolog | |
| 52-53 | CT1465 | CG12007 | | NP_004572.1\| (NM_004581) | 278 |
| | | geranylgeranyltransferase, | | Rab geranylgeranyltransferase, | |
| | | alpha subunit | | alpha subunit [Homo sapiens] | |
| 54-55 | CT5438 | CG12079 NADH | complex1_30 Kd | AAD40386.1\| (AF100743) | 323 |
| | | dehydrogenase | | NADH-Ubiquinone reductase | |
| | | (ubiquinone) | | [Homo sapiens] | |
| 56-57 | CT43008 | CG12085 pUbsf DPUF68 | RBD, RNP_1, rrm | NP_525123.1\| (NM_080384) | 1037 |
| | | Puf60 polyU binding | | poly-U-binding splicing factor | |
| | | splicing factor, poly(U) | | | |
| | | binding involved in | | | |
| | | mRNA splicing | | | |
| 58-59 | CT5902 | CG12093 unknown | CRYSTALLIN_BETAGAMMA | NP_499515.1\| (NM_067114) | 137 |
| | | | | Y41C4A.8.p [Caenorhabditis | |
| | | | | elegans] | |
| 60-61 | CT6734 | CG12113 unknown | ATP_GTP_A | AAH08013 (BC008013) Similar | 498 |
| | | | | to CG12113 gene product | |
| | | | | [Homo sapiens] | |
| 62-63 | CT7760 | CG12135 c12.1 unknown | | AF110775_1 (AF110775) | 252 |
| | | | | adrenal gland protein AD-002 | |
| | | | | [Homo sapiens] | |
| 64-65 | CT9355 | CG12181 Sgs4 sgs-4 | | Mus musculus Sap62 | |
| | | salivary gland secretion | | MGI: 104912 | |
| | | protein 4, pupal glue | | | |
| | | protein | | | |
| 66-67 | CT12665 | CG12225 Spt6 spt6, | S1 | Caenorhabditis elegans | |
| | | promoter-associated | | T04A8.14 WP: CE13120 | |
| | | pausing and | | | |
| | | transcriptional elongation | | | |
| 68-69 | CT13424 | CG12238 'probable | | NP_060758.1\| (NM_018288) | 222 |
| | | transcription factor | | hypothetical protein FLJ10975 | |
| | | | | [Homo sapiens] | |
| 70-71 | CT14932 | CG12251 AQP AQP | | XP_059490.1\| (XM_059490) | 62.4 |
| | | aquaporin, water channel | | hypothetical protein XP_059490 | |
| | | | | [Homo sapiens] | |
| 72-73 | CT23511 | CG12348 Sh open | | | |
| | | rectifying potassium | | | |
| | | channel, shaker | | | |
| 74-75 | CT32757 | CG12482 unknown | | NP_076113.1\| (NM_023624) | 40.8 |
| | | | | lecithin-retinol acyltransferase | |
| | | | | [Mus musculus] | |
| 76-77 | CT33237 | CG12497 | LDLRA_1, LDLRA_2, | CAC86027.1\| (AJ313389) tsetse | 90.9 |
| | | EG: BACR25B3.2 low- | LDLRECEPTOR, NLS_BP, | EP protein [Glossina morsitans | |
| | | density lipoprotein | PRO_RICH, ldl_recept_a | morsitans] | |
| | | receptor-like | | | |
| 78-79 | CT33996 | CG12537 unknown | | AAK31375.1\|AC084329_1 | 116 |
| | | | | (AC084329) ppg3 [Leishmania | |
| | | | | major] | |
| 80-81 | CT34671 | CG12600 unknown | WW_rsp5_WWP | AF213258_1 (AF213258) | 56.2 |
| | | | | membrane-associated guanylate | |
| | | | | kinase-related MAGI-3 [Mus | |
| | | | | musculus] | |
| 82-83 | CT2591 | CG1265 unknown | | XP_059471.1\| (XM_059471) | 67.8 |
| | | | | similar to MANNOSE-P- | |
| | | | | DOLICHOL UTILIZATION | |
| | | | | DEFECT 1 | |
| 84-85 | CT35764 | CG12701 unknown | NLS_BP, PRO_RICH, | NM_078717) kismet | 117 |
| | | | ZINC_FINGER_C2H2, | [Drosophila melanogaster] | |
| | | | ZINC_FINGER_C2H2_2, zf- | | |
| | | | C2H2 | | |
| 86-87 | CT28931 | CG12750 nucampholin, | RNA binding | (AB046824) KIAA1604 protein | 833 |
| | | transcription factor? | | [Homo sapiens] | |
| 88-89 | CT32253 | CG13034 unknown | | (AC084329) ppg3 [Leishmania | 94.4 |
| | | | | major] | |
| 90-91 | CT32701 | CG13372 EG: 171D11.6 | | none | |
| | | unknown | | | |
| 92-93 | CT40992 | CG13372 EG: 171D11.6 | | none | |
| | | unknown | | | |
| 94-95 | CT32721 | CG13380 unknown | | NP_499428.1\| (NM_067027) | 43.5 |
| | | | | W09D6.5.p [Caenorhabditis | |
| | | | | elegans] | |
| 96-97 | CT33014 | CG13620 unknown | CYTOCHROME_C, NLS_BP, | Caenorhabditis elegans ‘similar | |
| | | | ZINC_FINGER_C2H2, | to Zinc finger, C2H2 type | |
| | | | ZINC_FINGER_C2H2_2, zf- | | |
| | | | C2H2 | | |
| 98-99 | CT33019 | CG13625 histone | NLS_BP | NP_498982.1\| (NM_066581) | 265 |
| | | protein? | | R08D7.1.p [Caenorhabditis | |
| | | | | elegans] | |
| 100-101 | CT33241 | CG13760 | Cysteine proteinases | (AK054681) unnamed protein | 144 |
| | | EG: BACR25B3.6 | | product [Homo sapiens] | |
| | | unknown | | | |
| 102-103 | CT33317 | CG13818 unknown | ATP_GTP_A | T26047 hypothetical protein | 39.3 |
| | | | | W01C8.5 - Caenorhabditis | |
| | | | | elegans | |
| 104-105 | CT3228 | CG1405 cg1405 ATP | HELICASE, helicase_C | XP_008088.1\| (XM_008088) | 825 |
| | | dependent helicase | | pre-mRNA splicing factor Prp16 | |
| | | | | [Homo sapiens] | |
| 106-107 | CT33819 | CG14206 structural | | AF400207_1 (AF400207) | 225 |
| | | protein of ribosome | | ribosomal protein S10 | |
| | | | | [Spodoptera frugiperda] | |
| 108-109 | CT3352 | CG1422 p115 vesicular | | P41541\|VDP_BOVIN General | 725 |
| | | transporter, membrane | | vesicular transport factor p115 | |
| | | docking | | | |
| 110-111 | CT33841 | CG14226 CT33841 | fn3 | NP_075214.1\| (NM_022925) | 93.6 |
| | | protein tyrosine | | protein tyrosine phosphatase, | |
| | | phosphatase | | receptor type, Q [Rattus | |
| 112-113 | CT34063 | CG14411 protein | CRYSTALLIN_BETAGAMMA | AAK26171.1\| (AY028703) | 211 |
| | | phosphatase | | phosphatidylinositol-3 phosphate | |
| | | | | 3-phosphatase adaptor | |
| 114-115 | CT3509 | CG1448 inx3 innexin 3 | | Q9XYN1\|INX2_SCHAM | 332 |
| | | | | Innexin Inx2 (Innexin-2) (G- | |
| | | | | Inx2) | |
| 116-117 | CT34434 | CG14656 unknown | | NP_542443.1\| (NM_080712) | 122 |
| | | | | tty-P1 [Drosophila | |
| | | | | melanogaster] | |
| 118-119 | CT34588 | CG14778 integral | | (AE003604) CG2022 gene | 179 |
| | | peroxisomal membrane | | product [Drosophila | |
| | | | | melanogaster] | |
| 120-121 | CT43287 | CG14779 EG: 80H7.2 | Tubulin-beta mRNA | none | |
| | | tubulin-beta mRNA | autoregulation signal domain | | |
| | | autoregulation signal | | | |
| | | protein | | | |
| 122-123 | CT34589 | CG14779 EG: 80H7.2 | Tubulin-beta mRNA | none | |
| | | tubulin-beta mRNA | autoregulation signal domain | | |
| | | autoregulation signal | | | |
| | | protein | | | |
| 124-125 | CT34599 | CG14789 | AA_TRNA_LIGASE_I | AF455270_1 (AF455270) | 261 |
| | | EG: BACN32G11.6 | | C21ORF80 [Mus musculus] | |
| | | Aminoacyl-transfer RNA | | | |
| | | synthetases class-I | | | |
| | | signature protein | | | |
| 126-127 | CT34602 | CG14792 sta Laminin- | RIBOSOMALS2, | (AB032438) stubarista | 410 |
| | | receptor Stubarista, | RIBOSOMAL_S2_1, | [Drosophila erecta] | |
| | | protein biosynthesis Rp40 | RIBOSOMAL_S2_2, | | |
| | | | Ribosomal_S2 | | |
| 128-129 | CT34626 | CG14813 delta; COP | ATP_GTP_A: ATP/GTP- | NP_001646.2\| (NM_001655) | 585 |
| | | coatomer complex COPI | binding site motif A (P-loop) | archain; coatomer protein delta- | |
| | | delta-COP subunit delta | protein | COP [Homo sapiens] | |
| 130-131 | CT34665 | CG14849 unknown | | none | |
| 132-133 | CT3729 | CG1489 Pros45 sug1, | AAA, ATP_GTP_A | P54814\|PRS8_MANSE 26S | 727 |
| | | multicatalytic | | PROTEASE REGULATORY | |
| | | endopeptidase regulator, | | SUBUNIT 8 (18-56 PROTEIN) | |
| | | multicatalytic | | | |
| | | endopeptidase,, | | | |
| | | proteasome ATPase, | | | |
| | | preoteolysis and | | | |
| | | pepitolysis | | | |
| 134-135 | CT34842 | CG14991 unknown | BAND_41_3, PH_DOMAIN | XP_051693.1\| (XM_051693) | 635 |
| | | | | mitogen inducible 2 [Homo | |
| | | | | sapiens] | |
| 136-137 | CT34979 | CG15104 topoisomerase | | NP_055023.1\| (NM_014208) | 102 |
| | | I-binding RS protein’ | | dentin sialophosphoprotein; | |
| | | | | dentin phosphophoryn; | |
| 138-139 | CT3955 | CG1530 unknown | PRO_RICH | XP_092523.1\| (XM_092523) | 230 |
| | | | | hypothetical protein XP_092523 | |
| | | | | [Homo sapiens] | |
| 140-141 | CT35308 | CG15321 unknown | | none | |
| 142-143 | CT35676 | CG15560 putative cell | | NP_499205.1\| (NM_066804) | 170 |
| | | membrane-associated | | Transmembrane and sushi | |
| | | mucin | | domain [Caenorhabditis elegans] | |
| 144-145 | CT30180 | CG15811 Rop rop, ‘Ras | Sec1 | NP_037170.1\| (NM_013038) | 756 |
| | | opposite | | syntaxin binding protein 1 | |
| | | | | [Rattus norvegicus] | |
| 146-147 | CT34113 | CG15896 unknown | | NP_055487.1\| (NM_014672) | 182 |
| | | | | KIAA0391 gene product [Homo | |
| | | | | sapiens] | |
| 148-149 | CT34115 | CG15898 unknown | | NP_078828.1\| (NM_024552) | 47.8 |
| | | | | hypothetical protein FLJ12089 | |
| | | | | [Homo sapiens] | |
| 150-151 | CT4708 | CG1683 Ant2 Ant2, | ADPTRNSLCASE, | (AF218587) ADP/ATP | 485 |
| | | ADP/ATP translocase. | MITOCARRIER, | translocase [Lucilia cuprina] | |
| | | Adenine nucleotide | MITOCH_CARRIER, mito_carr | | |
| | | translocase 2, ATP/ADP | | | |
| | | antiporter | | | |
| 152-153 | CT37506 | CG16903 EG: 67A9.2 | | NP_446114.1\| (NM_053662) | 411 |
| | | non-specific RNA | | cyclin L [Rattus norvegicus] | |
| | | polymerase II | | | |
| | | transcription factor | | | |
| 154-155 | CT35131 | CG16916 Rpt3 p48A, 26S | AAA, CLPPROTEASEA | PRS6_MANSE 26S | 681 |
| | | proteasome regulatory | | PROTEASE REGULATORY | |
| | | complex subunit p48A | | SUBUNIT 6B (ATPASE MS73) | |
| 156-157 | CT4802 | CG1696 unknown | | NP_056158.1\| (NM_015343) | 341 |
| | | | | hypothetical protein [Homo | |
| | | | | sapiens] | |
| 158-159 | CT43084 | CG1697 rho-4 rho-4 Rho- | | Rattus norvegicus ‘rhomboid- | |
| | | related [10C6] rhomboid-4 | | related protein’ | |
| | | | | EMBL: Y17258 | |
| 160-161 | CT4810 | CG1698 unknown | | none | |
| 162-163 | CT4826 | CG1703 ATP-binding | ABC_TRANSPORTER, | (AF293383) ABC50 [Rattus | 802 |
| | | cassette (ABC) transporter | ABC_tran, ATP_GTP_A, | norvegicus] | |
| | | | ATP_GTP_A2, DA_BOX, | | |
| | | | NLS_BP | | |
| 164-165 | CT35402 | CG17252 BCL7-like | | (NM_001707) B-cell | 94.4 |
| | | BCL7-like | | CLL/lymphoma 7B [Homo | |
| | | | | sapiens] | |
| 166-167 | CT21145 | CG17309 CSK CSK, | PROTEIN_KINASE_ATP, | AAH18394 (BC018394) c-src | 462 |
| | | involved in protein | PROTEIN_KINASE_DOM, | tyrosine kinase [Mus musculus] | |
| | | phosphorylation | PROTEIN_KINASE_TYR, | | |
| | | | SH2, SH2DOMAIN, | | |
| | | | TYRKINASE, pkinase | | |
| 168-169 | CT5050 | CG1740 Ntf-2 NTF-2, | NTF2_DOMAIN | (NM_059921) nuclear transport | 127 |
| | | protein carrier involved in | | factor 2 like [Caenorhabditis | |
| | | protein-nucleus import | | | |
| 170-171 | CT5086 | CG1746 anon- | ATP-synt_C, ATPASEC, | Q9U505\|ATPC_MANSE ATP | 177 |
| | | EST: Posey224 hydrogen- | ATPASE_C | synthase subunit C, | |
| | | transporting ATP | | mitochondrial precursor (Lipid- | |
| | | synthase/enzyme, | | binding | |
| | | hydrogen-transporting | | | |
| | | two-sector ATPase | | | |
| 172-173 | CT34491 | CG17734 unknown | | NP_062788.1\| (NM_019814) | 82.4 |
| | | | | hypoxia induced gene 1 [Mus | |
| | | | | musculus] | |
| 174-175 | CT39345 | CG17766 EG: 86E4.3 | WD40, WD40_REGION | AF188123_1 (AF188123) TGF- | 1160 |
| | | heterotrimeric G-protein | | beta resistance-associated | |
| | | GTPase | | protein TRAG [Mus musculus] | |
| 176-177 | CT39414 | CG17791 sqd | RBD, rrm; Eukaryotic putative | Homo sapiens ‘heterogeneous | |
| | | heterogeneous-nuclear- | RNA-binding region RNP-1 | nuclear ribonucleoprotein D’ | |
| | | ribonucleoprotein-87Fb | signature, RRM-motif protein, | EMBL: AF026126 | |
| | | RNA-binding protein 3, | RRM-motif protein | | |
| | | Squid | | | |
| 178-179 | CT39758 | CG17871 Or71a tracheal | | none | |
| | | gasfilling mutant1b, | | | |
| | | Or71a, odorant receptor | | | |
| 180-181 | CT40282 | CG18009 Trf2 TATA box | | (AB024489) TBP-like protein | 210 |
| | | binding protein-related | | [Gallus gallus] | |
| | | factor 2 | | | |
| 182-183 | CT5456 | CG1826 product | BTB, NLS_BP, | (AB067467) KIAA1880 protein | 595 |
| | | involved in developmental | PROTEIN_SPLICING | [Homo sapiens] | |
| | | processes | | | |
| 184-185 | CT41472 | CG18282 Ubiquitin-like | | I45964 polyubiquitin - bovine | 431 |
| | | | | (fragment) | |
| 186-187 | CT42468 | CG18578 Ugt86Da UDP- | | none | |
| | | glucuronosyltransferase | | | |
| 188-189 | CT13908 | CG18734 Fur2 furin | | T43251 furin (EC 3.4.21.75) - | 1753 |
| | | | | fall armyworm | |
| 190-191 | CT5890 | CG1908 unknown | NLS_BP | none | |
| 192-193 | CT5932 | CG1915 sls sallimus, | AA_TRNA_LIGASE_II_1, | Gallus gallus ‘connectin/titin’ | |
| | | myosin light chain kinase | ATP_GTP_A, NLS_BP, SH3, | EMBL: D83390 | |
| | | | fn3, ig | | |
| 194-195 | CT6007 | CG1937 involved in cell | | (AF317634) HRD1 [Homo | 545 |
| | | growth and maintenance | | sapiens] | |
| 196-197 | CT5951 | CG1938 Dlic2 Dlic2, | ATP_GTP_A | (AF317841) cytoplasmic dynein | 399 |
| | | motor which is a | | light-intermediate chain 1 | |
| | | component of the | | [Xenopus | |
| | | microtubule associated | | | |
| | | protein | | | |
| 198-199 | CT6352 | CG1994 similar to | ATP_GTP_A | (AB051496) KIAA1709 protein | 1013 |
| | | Achlya ambisexualis | | [Homo sapiens] | |
| | | antheridiol steroid | | | |
| | | receptor | | | |
| 200-201 | CT6373 | CG2003 high affinity | transporter | Homo sapiens ‘Na/PO4 | |
| | | inorganic | | cotransporter’ gi: 4885441 | |
| | | phosphate:sodium | | | |
| | | symporter | | | |
| 202-203 | CT4336 | CG2151 Trxr-1 NOT | FADPNR, HGRDTASE, | (U88187) glutathione reductase | 753 |
| | | glutathione reductase | NAD_BINDING, | family member [Musca | |
| | | (NADPH) (EC: 1.6.4.2) | PNDRDTASEI, | domestica] | |
| | | involved in thioredoxin | PYRIDINE_REDOX_1, | | |
| | | reduction | pyr_redox | | |
| 203-205 | CT6738 | CG2165 BEST: CK01140 | | (NM_053311) ATPase, Ca++ | 1262 |
| | | calcium-transporting | | transporting, plasma membrane | |
| | | ATPase-like | | 1 [Rattus | |
| 206-207 | CT5965 | CG2184 Mlc2 muscle- | EF_HAND, EF_HAND_2, | MLR5_FELCA Superfast | 130 |
| | | specific myosin regulatory | efhand | myosin regulatory light chain 2 | |
| | | light chain Mlc2, involved | | (MYLC2) | |
| | | in cell motility | | | |
| 208-209 | CT7322 | CG2222 unknown | | none | |
| 210-211 | CT7705 | CG2309 ERK7 protein | | YPC2_CAEEL Putative | 392 |
| | | kinase, protein | | serine/threonine-protein kinase | |
| | | serine/threonine kinase | | C05D10.2 in chromosome III | |
| 212-213 | CT8341 | CG2520 lap lap, | ENTH | (AF182339) clathrin assembly | 502 |
| | | chaperone | | protein AP180 [Loligo pealei] | |
| 214-215 | CT9021 | CG2666 CS-1 CS-1, | | (AF221067) chitin synthase 1 | 2770 |
| | | enzyme/chitin synthase | | [Lucilia cuprina] | |
| 216-217 | CT9593 | CG2829 | NLS_BP, PFKB_KINASES_1, | (AB004884) PKU-alpha [Homo | 520 |
| | | BcDNA: GH07910 protein | PROTEIN_KINASE_ATP, | sapiens] | |
| | | kinase, protein | PROTEIN_KINASE_DOM, | | |
| | | serine/threonine kinase | PROTEIN_KINASE_ST, | | |
| | | | PRO_RICH, pkinase | | |
| 218-219 | CT9754 | CG2849 Rala Ral, RAS | ATP_GTP_A, PRENYLATION, | (XM_035787) similar to Ras- | 304 |
| | | small monomeric GTPase, | RASTRNSFRMNG, ras | related protein RAL-A [Homo | |
| | | regulates developmental | | sapiens] | |
| | | cell shape changes | | | |
| | | through the JNK pathway | | | |
| 220-221 | CT9660 | CG2829 | NLS_BP, PFKB_KINASES_1, | (AB004884) PKU-alpha [Homo | 520 |
| | | BcDNA: GH07910 protein | PROTEIN_KINASE_ATP, | sapiens] | |
| | | kinase, protein | PROTEIN_KINASE_DOM, | | |
| | | serine/threonine kinase | PROTEIN_KINASE_ST, | | |
| | | | PRO_RICH, pkinase | | |
| 222-223 | CT6171 | CG2968 hydrogen- | | P35434\|ATPD_RAT ATP | 142 |
| | | transporting ATP | | synthase delta chain, | |
| | | synthase, coupling factor | | mitochondrial precursor | |
| | | CF(0), delta-chain | | | |
| 224-225 | CT10206 | CG3034 EG: BACR7A4.6 | | (Y15172) surfeit protein 5 | 183 |
| | | similar to Surf5b [Homo | | [Takifugu rubripes] | |
| | | sapiens | | | |
| 226-227 | CT41361 | CG3071 EG: 25E8.3 | Trp-Asp (WD) repeats signature | T40471 probable Trp-Asp repeat | 273 |
| | | involved in retrograde | protein | protein - fission yeast | |
| | | (Golgi to ER) transport | | | |
| | | which is putatively a | | | |
| | | component of the | | | |
| | | coatomer | | | |
| 228-229 | CT9947 | CG3071 EG: 25E8.3 | Trp-Asp (WD) repeats signature | T40471 probable Trp-Asp repeat | 273 |
| | | involved in retrograde | protein | protein - fission yeast | |
| | | (Golgi to ER) transport | | | |
| | | which is putatively a | | | |
| | | component of the | | | |
| | | coatomer | | | |
| 230-231 | CT10723 | CG3201 Mlc-c Mlc-c, | EF_HAND, EF_HAND_2, | Homo sapiens ‘MYOSIN | |
| | | alkali light chain of non- | efhand | LIGHT CHAIN ALKALI, | |
| | | muscle myosin-II, | | SMOOTH-MUSCLE | |
| | | cytoskeleton organization | | ISOFORM (MLC3SM) | |
| | | and biogenesis | | (LC17B) (LC’ SWP: P24572 | |
| 232-233 | CT11063 | CG3313 transcription | NLS_BP, WD40, | (AB067479) KIAA1892 protein | 293 |
| | | factor | WD40_REGION | [Homo sapiens] | |
| 234-235 | CT11487 | CG3415 estradiol 17 | ADH_SHORT, GDHRDH, | (NM_000414) hydroxysteroid | 613 |
| | | beta-dehydrogenase | THIOL_PROTEASE_HIS, | (17-beta) dehydrogenase 4 | |
| | | | adh_short | [Homo sapiens] | |
| 236-237 | CT11597 | CG3446 unknown | | (AJ316011) mitochondrial | 78.6 |
| | | | | NADH: ubiquinone | |
| | | | | oxidoreductase B16.6 | |
| 238-239 | CT11623 | CG3455 Rpt4 Rpt4, | | Manduca sex ‘26S proteasome | |
| | | endopeptidase, | | regulatory ATPase subunit 10b | |
| | | multicatalytic | | (S10b)’ EMBL: AJ223384 | |
| | | endopeptidase regulator, | | | |
| | | multicatalytic | | | |
| | | endopeptidase, | | | |
| | | proteasome ATPase | | | |
| 240-241 | CT11966 | CG3560 anon- | | 1BCC\|F Chain F, Cytochrome | 150 |
| | | EST: Posey167 NADH | | Bc1 Complex From Chicken | |
| | | dehydrogenase | | | |
| 242-243 | CT12417 | CG3703 | | (NM_075735) T19D7.4.p | 251 |
| | | EG: BACR7A4.15 | | [Caenorhabditis elegans] | |
| | | cytoskeleton organization | | | |
| | | and biogenesis | | | |
| 244-245 | CT12443 | CG3715 Shc dShc, SHC- | | S25776 transforming protein | 267 |
| | | adaptor protein, protein | | (SHC) - human | |
| | | kinase putatively involved | | | |
| | | in cell growth and | | | |
| | | maintenance | | | |
| 246-247 | CT12517 | CG3747 Eaat1 Eaat1, | plasma membrane | (AF330257) glutamate | 402 |
| | | glutamate transporter, | | transporter [Mus musculus] | |
| | | Excitatory amino acid | | | |
| | | transporter 1 | | | |
| 248-249 | CT12871 | CG3861 citrate (SI)- | CITRATE_SYNTHASE, | P00889\|CISY_PIG CITRATE | 674 |
| | | synthase | CITRTSNTHASE, citrate_synt | SYNTHASE, | |
| | | | | MITOCHONDRIAL | |
| | | | | PRECURSOR | |
| 250-251 | CT12909 | CG3874 nucleotide-sugar | | (NM_015139) UDP-glucuronic | 361 |
| | | transporter-like | | acid/UDP-N- | |
| | | | | acetylgalactosamine dual | |
| 252-253 | CT13223 | CG3981 Unc-76 Dunc- | | (NM_005102) zygin 2; | 197 |
| | | 76, signal transducer | | fasciculation and elongation | |
| | | involved in axon cargo | | protein zeta 2; | |
| | | transport | | | |
| 254-255 | CT4722 | CG4013 Smr Smrter | ANTIFREEZEI, myb_DNA- | NCR2_MOUSE NUCLEAR | 275 |
| | | SMRT-related ecdysone | binding | RECEPTOR CO-REPRESSOR | |
| | | receptor-interacting factor | | 2 (N-COR2) (SILENCING | |
| | | SANT domain protein, | | MEDIATOR OF | |
| | | transcription corepressor | | | |
| 256-257 | CT13458 | CG4094 fumarate | DCRYSTALLIN, | (NM_017005) fumarate | 512 |
| | | hydratase, enzyme | FUMARATE_LYASES, | hydratase [Rattus norvegicus] | |
| | | involved in main | FUMRATELYASE, lyase_1 | | |
| | | pathways of carbohydrate | | | |
| | | metabolism | | | |
| 258-259 | CT13690 | CG4129 | | (XM_043094) KIAA0061 | 325 |
| | | BcDNA: LD21623 | | protein [Homo sapiens] | |
| | | unknown | | | |
| 260-261 | CT5938 | CG4147 Hsc70-3 Hsc70- | ER_TARGET, | (AB016836) heat shock 70 kD | 1159 |
| | | 3, Heat shock protein | HEATSHOCK70, HSP70, | protein cognate [Bombyx mori] | |
| | | cognate 3, involved in | HSP70_1, HSP70_2, HSP70_3 | | |
| | | stress response | | | |
| 262-263 | CT13852 | CG4202 Sas10 Sas10 | | (NM_023054) disrupter of | 259 |
| | | | | silencing SAS10 [Mus | |
| | | | | musculus] | |
| 264-265 | CT14019 | CG4300 spermidine | SAM_BIND | (AJ009865) spermine synthase | 276 |
| | | synthase | | [Takifugu rubripes] | |
| 266-267 | CT14119 | CG4300 spermidine | SAM_BIND | (AJ009865) spermine synthase | 276 |
| | | synthase | | [Takifugu rubripes] | |
| 268-269 | CT13914 | CG4317 Mipp2 Mipp2, | CYTOCHROME_B_QO | Mus musculus ‘multiple inositol | |
| | | multiple inositol- | | polyphosphate phosphatase’ | |
| | | polyphosphate | | EMBL: AF046908 | |
| | | phosphatase 2 | | | |
| 270-271 | CT14464 | CG4453 transporter, an | ZF_RANBP, zf-RanBP | 14578 nucleoporin Nup153 | 300 |
| | | endopeptidase involved in | | homolog - African clawed frog | |
| | | behavior which is a | | (fragment) | |
| | | component of the nucleus | | | |
| 272-273 | CT14586 | CG4481 Glu-RIB ion | ANF_receptor, | Mus musculus ‘glutamate | |
| | | channel-alpha-amino-3- | CHANNEL_PORE_K, | receptor channel a3 subunit’ | |
| | | hydroxy-5-methyl-4- | NLS_BP, SBP_GLUR, lig_chan | EMBL: AB022342 | |
| | | isoxazole propionate | | | |
| | | selective glutamate | | | |
| | | receptor; ionotropic | | | |
| | | glutamate receptor | | | |
| 274-275 | CT14874 | CG4590 inx2 inx2, | Innexin | Schistocerca americana | |
| | | neurotransmitter | | ‘innexin-2’ EMBL: 115854_1 | |
| | | transporter, Dm-inx pas | | | |
| | | related protein 33 | | | |
| 276-277 | CT15952 | CG4974 dally NOT cell | Glypican | (NM_004466) glypican 5 | 186 |
| | | adhesion molecule; | | [Homo sapiens] | |
| | | heparin sulfate | | | |
| | | proteoglycan; Dally | | | |
| 278-279 | CT16489 | CG5147 unknown | | none | |
| 280-281 | CT16663 | CG5208 | | none | |
| | | BcDNA: LD27979 | | | |
| | | unknown | | | |
| 282-283 | CT17394 | CG5485 high affinity | | (AF349043) sulfate anion | 340 |
| | | sulfate permease, sulfate | | transporter-1 [Mus musculus] | |
| | | transporter | | | |
| 284-285 | CT17382 | CG5486 Ubp64E | | (NM_063285) ubiquitin | 358 |
| | | Ubiquitin-specific | | carboxyl-terminal hydrolase | |
| | | protease 64E | | [Caenorhabditis | |
| 286-287 | CT17448 | CG5505 endopeptidase, | UCH-1, UCH-2, UCH_2_1, | (XM_027039) KIAA1453 | 254 |
| | | ubiquitin-specific | UCH_2_2, UCH_2_3 | protein [Homo sapiens] | |
| | | protease, involved in | | | |
| | | process of | | | |
| | | deubiquitylation | | | |
| 288-289 | CT17938 | CG5684 non-specific | | Q9UIV1\|CNO7_HUMAN | 376 |
| | | RNA polymerase II | | CCR4-NOT transcription | |
| | | transcription factor | | complex, subunit 7 (CCR4- | |
| | | | | associated factor | |
| 290-291 | CT17971 | CG5722 NPC1 dmNPC1, | 5TM_BOX, NLS_BP | (NM_000271) Niemann-Pick | 1061 |
| | | transmembrane receptor | | disease, type C1 [Homo sapiens] | |
| 292-293 | CT18192 | CG5797 cytoskeletal | PRO_RICH | (AB051482) KIAA1695 protein | 541 |
| | | binding protein | | [Homo sapiens] | |
| 294-295 | CT18619 | CG5939 Prm Para, | NLS_BP | (AF317670) paramyosin | 989 |
| | | Paramyosin, structural | | [Sarcoptes scabiei] | |
| | | protein of muscle, motor | | | |
| 296-297 | CT18969 | CG6058 Ald fructose- | ALDOLASE_CLASS_I, | Mus musculus Aldo1 | |
| | | bisphosphate aldolase, | NLS_BP, glycolytic_enzy | MGI: 87994 | |
| | | involved in process of | | | |
| | | glycolysis | | | |
| 298-299 | CT19788 | CG6335 histidine--tRNA | AA_TRNA_LIGASE_II_1, | (NM_008214) histidyl tRNA | 641 |
| | | ligase | AA_TRNA_LIGASE_II_2, | synthetase [Mus musculus] | |
| | | | WHEP-TRS, tRNA-synt_2b | | |
| 300-301 | CT19850 | CG6367 serine-type | | (AF053921) trypsin-like serine | 163 |
| | | endopeptidase | | protease [Ctenocephalides felis] | |
| 302-303 | CT19962 | CG6400 unknown | BROMODOMAIN, | Q9NSI6\|WDR9_HUMAN WD- | 916 |
| | | | BROMODOMAIN_2, | REPEAT PROTEIN 9 | |
| | | | GPROTEINBRPT, NLS_BP, | | |
| | | | WD40, WD40_REGION, | | |
| | | | WD_REPEATS, bromodomain | | |
| 304-305 | CT20122 | CG6470 unknown | ZINC_FINGER_C2H2, | none | |
| | | | ZINC_FINGER_C2H2_2, zf- | | |
| | | | C2H2 | | |
| 306-307 | CT20269 | CG6513 signal | | (NM_019561) endosulfine | 91.3 |
| | | transduction | | alpha; alpha-endosulfine [Mus | |
| | | | | musculus] | |
| 308-309 | CT21021 | CG6774 tracheal | | (NM_023037) hypothetical | 1006 |
| | | gasfilling mutant | | protein CG003 [Homo sapiens] | |
| 310-311 | CT21292 | CG6874 unknown | | none | |
| 312-313 | CT43217 | CG6928 Sulfate | Sulfate_transp | | |
| | | transporter | | | |
| 314-315 | CT21476 | CG6930 unknown | NLS_BP, | Caenorhabditis elegans ‘contains | |
| | | | ZINC_FINGER_C2H2, | strong similarity to a C2H2-type | |
| | | | ZINC_FINGER_C2H2_2, zf- | zinc finger’ EMBL: AF000194 | |
| | | | C2H2 | | |
| 316-317 | CT21525 | CG6946 RNA binding | RBD, rrm | Rattus norvegicus | |
| | | | | ‘ribonucleoprotein F’ | |
| | | | | EMBL: AB022209 | |
| 318-319 | CT21704 | CG7014 structural | RIBOSOMAL_S7, | (NM_001009) ribosomal protein | 347 |
| | | protein of ribosome, | Ribosomal_S7 | S5; 40S ribosomal protein S5 | |
| | | Process protein | | [Homo | |
| | | biosynthesis | | | |
| 320-321 | CT22195 | CG7187 DNA binding | | (AY026310) single stranded | 351 |
| | | | | DNA binding protein-1 [Homo | |
| | | | | sapiens] | |
| 322-323 | CT22253 | CG7215 ubiquitin | UBIQUITIN_2, ubiquitin | P21126\|UBLG_MOUSE | 75.5 |
| | | | | Ubiquitin-like protein GDX | |
| | | | | (Ubiquitin-like protein 4) | |
| 324-325 | CT22861 | CG7434 RpL22 ribosomal | ANTIFREEZEI | (AF400188) ribosomal protein | 165 |
| | | protein L22 | | L22 [Spodoptera frugiperda] | |
| 326-327 | CT23083 | CG7552 unknown | ATP_GTP_A, | Homo sapiens ‘65 KD YES- | |
| | | | WW_DOMAIN_1, | ASSOCIATED PROTEIN | |
| | | | WW_DOMAIN_2, | (YAP65)’ SWP: P46937 | |
| | | | WW_rsp5_WWP | | |
| 328-329 | CT23596 | CG7757 similarity to | NLS_BP | (NM_004698) U4/U6-associated | 520 |
| | | U4/U6-associated RNA | | RNA splicing factor [Homo | |
| | | splicing factor | | sapiens] | |
| 330-331 | CT23626 | CG7770 cochaperonin in | | (NM_010385) H2-K region | 106 |
| | | process of ‘de novo’ | | expressed gene 2 [Mus | |
| | | protein folding | | musculus] | |
| 332-333 | CT23882 | CG7901 PP2A-B′ protein | ANTIFREEZEI | Mus musculus ‘protein | |
| | | phosphatase, protein | | phosphatase 2A B′a3 regulatory | |
| | | phosphatase type 2A | | subunit’ EMBL: U37353 | |
| | | regulator | | | |
| 334-335 | CT41698 | CG7958 unknown | | (AB033050) KIAA1224 protein | 427 |
| | | | | [Homo sapiens] | |
| 336-337 | CT23982 | CG7958 unknown | | (AB033050) KIAA1224 protein | 427 |
| | | | | [Homo sapiens] | |
| 338-339 | CT23998 | CG7983 guanylate kinase | PRO_RICH | (AF411837) transcription | 214 |
| | | | | repressor p66 [Mus musculus] | |
| 340-341 | CT24094 | CG8031 unknown | | (BC013819) CGI-27 protein | 394 |
| | | | | [Mus musculus] | |
| 342-343 | CT24122 | CG8037 ELL, DNA- | | Gallus gallus ‘OCCLUDIN’ | |
| | | directed RNA polymerase | | SWP: Q91049 | |
| | | III; | | | |
| 344-345 | CT24346 | CG8148 timeout timeout | | (NM_003920) timeless | 149 |
| | | | | (Drosophila) homolog [Homo | |
| | | | | sapiens] | |
| 346-347 | CT24393 | CG8189 ATPsyn-b | Acetyltransf | (AF187862) ATP synthase | 213 |
| | | ATPsyn-b Fo-ATP | | subunit B [Xenopus laevis] | |
| | | synthase subunit b | | | |
| 348-349 | CT24437 | CG8231 T-complex | CHAPERONIN60, | O77622\|TCPZ_RABIT T- | 754 |
| | | protein 1, zeta-subunit, | TCOMPLEXTCP1, TCP1_1, | COMPLEX PROTEIN 1, ZETA | |
| | | chaperone | TCP1_2, TCP1_3, cpn60_TCP1 | SUBUNIT (TCP-1-ZETA) | |
| | | | | (CCT-ZETA) | |
| 350-351 | CT18257 | CG8322 ATPCL ATP- | SUCCINYL_COA_LIG_1, | (U18197) ATP: citrate lyase | 1555 |
| | | citrate (pro-S)-lyase | SUCCINYL_COA_LIG_2, | [Homo sapiens] | |
| | | | SUCCINYL_COA_LIG_3, | | |
| | | | ligase-CoA | | |
| 352-353 | CT24731 | CG8439 Cct5 Cct5, T- | | (XM_052313) chaperonin | 791 |
| | | complex Chaperonin 5, | | containing TCP1, subunit 5 | |
| | | tracheal gasfilling mutant | | (epsilon) [Homo | |
| 354-355 | CT24823 | CG8484 Transcription | ZINC_FINGER_C2H2, | (NM_058230) zinc finger | 167 |
| | | factor | ZINC_FINGER_C2H2_2, zf- | protein 354B [Homo sapiens] | |
| | | | C2H2 | | |
| 356-357 | CT25072 | CG8655 CDC receptor | AA_TRNA_LIGASE_II_2, | (AF005209) HsCdc7 [Homo | 216 |
| | | signaling protein | PROTEIN_KINASE_DOM, | sapiens] | |
| | | serine/threonine kinase | PROTEIN_KINASE_ST, | | |
| | | | pkinase | | |
| 358-359 | CT25274 | CG8759 Nacalpha; NAC | | Homo sapiens & agr | |
| | | protein alpha subunit, | | PIR: S49326 | |
| | | component of the nascent | | | |
| | | polypeptide-associated | | | |
| | | complex | | | |
| 360-361 | CT25472 | CG8870 endopeptidase, | ANTENNAPEDIA, | Caenorhabditis elegans ‘similar | |
| | | monophenol | CHYMOTRYPSIN, | to plasminogen and to trypsin- | |
| | | monooxygenase activator | TRYPSIN_CATAL, | like serine proteases’ | |
| | | | TRYPSIN_HIS, | EMBL: U29380 | |
| | | | TRYPSIN_SER, trypsin | | |
| 362-363 | CT25624 | CG8922 RpS5 Ribosomal | RIBOSOMAL_S7, | (Y12431) 5S ribosomal protein | 353 |
| | | protein S5 | Ribosomal_S7 | [Mus musculus] | |
| 364-365 | CT8969 | CG9165 enzyme, | PORPHBDMNASE, | P08397\|HEM3_HUMAN | 287 |
| | | hydroxymethylbilane | Porphobil_deam | PORPHOBILINOGEN | |
| | | synthase | | DEAMINASE | |
| | | | | (HYDROXYMETHYLBILANE | |
| | | | | SYNTHASE) (HMBS) | |
| 366-367 | CT27084 | CG9591 unknown | | (XM_043261) KIAA1698 | 116 |
| | | | | protein [Homo sapiens] | |
| 368-369 | CT27543 | CG9748 cap Belle, ATP | | 1705301A ATP dependent | 723 |
| | | dependent helicase | | RNA helicase [Xenopus laevis] | |
| 370-371 | CT27750 | CG9821 unknown | | none | |
| 372-373 | CT27796 | CG9901 Arp14D Actin- | ACTIN, ACTINS_ACT_LIKE, | P53488\|ARP2_CHICK ACTIN- | 678 |
| | | related protein 14D, arp2 | actin | LIKE PROTEIN 2 (ACTIN- | |
| | | | | LIKE PROTEIN ACTL) | |
| 374-375 | CT27906 | CG9910 katanin-80 | | (AF052433) katanin p80 subunit | 231 |
| | | katanin 80, microtubule | | [Strongylocentrotus purpuratus] | |
| | | severing which is a | | | |
| | | component of the katanin | | | |
| 376-377 | CT27940 | CG9924 transcription | BTB, MATH | (NM_003563) speckle-type POZ | 599 |
| | | factor | | protein [Homo sapiens] | |
| 378-379 | CT27993 | CG9946 eIF-2alpha; | NLS_BP, S1 | (NM_131800) eIF2 alpha | 376 |
| | | Eukaryotic initiation | | subunit [Danio rerio] | |
| | | factor 2A; translation | | | |
| | | initiation factor | | | |
| 380-381 | CT20536 | CG6606 unknown | ATPASE_ALPHA_BETA, | (AB020664) KIAA0857 protein | 122 |
| | | | ATP_GTP_A, C2, NLS_BP, | [Homo sapiens] | |
| | | | RECEPTOR_CYTOKINES_2 | | |

### DEFINITIONS

For clarity, certain terms used in the specification are defined and used as follows:

“Associated with/operatively linked” refer to two nucleic acid sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

A “chimeric construct” is a recombinant nucleic acid sequence in which a promoter or regulatory nucleic acid sequence is operatively linked to, or associated with, a nucleic acid sequence that codes for an mRNA or which is expressed as a protein, such that the regulatory nucleic acid sequence is able to regulate transcription or expression of the associated nucleic acid sequence. The regulatory nucleic acid sequence of the chimeric construct is not normally operatively linked to the associated nucleic acid sequence as found in nature.

Co-factor: natural reactant, such as an organic molecule or a metal ion, required in an enzyme-catalyzed reaction. A co-factor is e.g. NAD(P), riboflavin (including FAD and FMN), folate, molybdopterin, thiamin, biotin, lipoic acid, pantothenic acid and coenzyme A, S-adenosylmethionine, pyridoxal phosphate, ubiquinone, menaquinone. Optionally, a co-factor can be regenerated and reused.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

Complementary: “complementary” refers to two nucleotide sequences that comprise antiparallel nucleotide sequences capable of pairing with one another upon formation of hydrogen bonds between the complementary base residues in the antiparallel nucleotide sequences.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a protein also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a protein is implicit in each described sequence.

Furthermore, one of skill will recognize that individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine R), Lysine (K), Histidine (H); Acidic: Aspartic acid (D), Glutamic acid (E), Asparagine (N), Glutamine (Q). See also, Creighton (1984) Proteins, W. H. Freeman and Company. In addition, individual substitutions, deletions or additions which alter, add or delete a single amino acid or a small percentage of amino acids in an encoded sequence are also “conservatively modified variations.”

DNA Shuffling: DNA shuffling is a method to rapidly, easily and efficiently introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA encodes an enzyme modified with respect to the enzyme encoded by the template DNA, and preferably has an altered biological activity with respect to the enzyme encoded by the template DNA.

Enzyme/Protein Activity: means herein the ability of an enzyme (or protein) to catalyze the conversion of a substrate into a product. A substrate for the enzyme comprises the natural substrate of the enzyme but also comprises analogues of the natural substrate, which can also be converted, by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time.

Essential: an “essential” Drosophila melanogaster nucleotide sequence is a nucleotide sequence encoding a protein such as e.g. a biosynthetic enzyme, receptor, signal transduction protein, structural gene product, or transport protein that is essential to the growth or survival of the insect.

Expression Cassette: “Expression cassette” as used herein means a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus. In the case of a multicellular organism, such as an insect, the promoter can also be specific to a particular tissue or organ or stage of development.

Gene: the term “gene” is used broadly to refer to any segment of DNA associated with a biological function. Thus, genes include coding sequences and/or the regulatory sequences required for their expression. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

Heterologous/exogenous: The terms “heterologous” and “exogenous” when used herein to refer to a nucleic acid sequence (e.g. a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g. DNA) sequence is a nucleic acid (e.g. DNA) sequence naturally associated with a host cell into which it is introduced.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

Inhibitor: a chemical substance that inactivates the enzymatic activity of an enzyme (or protein) of interest The term “insecticide” is used herein to define an inhibitor when applied to an insect at any stage of development

Insecticide: a chemical substance used to kill or inhibit the growth or viability of insects at any stage of development.

Interaction: quality or state of mutual action such that the effectiveness or toxicity of one protein or compound on another protein is inhibitory (antagonists) or enhancing (agonists).

A nucleic acid sequence is “isocoding with” a reference nucleic acid sequence when the nucleic acid sequence encodes a polypeptide having the same amino acid sequence as the polypeptide encoded by the reference nucleic acid sequence.

An “isolated” nucleic acid molecule or an isolated enzyme is a nucleic acid molecule or enzyme that, by the hand of man, exists apart from its native environment and is therefore not a product of nature. An isolated nucleic acid molecule or enzyme may exist in a purified form or may exist in a non-native environment such as, for example, a recombinant host cell.

Mature Protein: protein that is normally targeted to a cellular organelle and from which the transit peptide has been removed.

Minimal Promoter: promoter elements, particularly a TATA element, that are inactive or that have greatly reduced promoter activity in the absence of upstream activation. In the presence of a suitable transcription factor, the minimal promoter functions to permit transcription.

Modified Enzyme Activity: enzyme activity different from that which naturally occurs in an insect (i.e. enzyme activity that occurs naturally in the absence of direct or indirect manipulation of such activity by man), which is tolerant to inhibitors that inhibit the naturally occurring enzyme activity.

Native: refers to a gene that is present in the genome of an untransformed insect cell.

Naturally occurring: the term “naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

Nucleic acid: the term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); Rossolini et al., Mol. Cell Probes 8: 91-98 (1994)). The terms “nucleic acid” or “nucleic acid sequence” may also be used interchangeably with gene, cDNA, and mRNA encoded by a gene.

“ORF” means open reading frame.

Purified: the term “purified,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

Two nucleic acids are “recombined” when sequences from each of the two nucleic acids are combined in a progeny nucleic acid. Two sequences are “directly” recombined when both of the nucleic acids are substrates for recombination. Two sequences are “indirectly recombined” when the sequences are recombined using an intermediate such as a cross-over oligonucleotide. For indirect recombination, no more than one of the sequences is an actual substrate for recombination, and in some cases, neither sequence is a substrate for recombination.

“Regulatory elements” refer to sequences involved in controlling the expression of a nucleotide sequence. Regulatory elements comprise a promoter operatively linked to the nucleotide sequence of interest and termination signals. They also typically encompass sequences required for proper translation of the nucleotide sequence.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

Substantially identical: the phrase “substantially identical,” in the context of two nucleic acid or protein sequences, refers to two or more sequences or subsequences that have at least 60%, preferably 80%, more preferably 90, even more preferably 95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In an especially preferred embodiment, the sequences are substantially identical over the entire length of the coding regions. Furthermore, substantially identical nucleic acid or protein sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis,), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403410 (1990). Software for performing BLAST analyses is publicly available through the National Center for Biotechnology Information on the world wide web at ncbi.nlm.nih.gov/. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

A further indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions.

The phrase “specifically (or selectively) binds to an antibody,” or “specifically (or selectively) immunoreactive with,” when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised to the protein with the amino acid sequence encoded by any of the nucleic acid sequences of the invention can be selected to obtain antibodies specifically immunoreactive with that protein and not with other proteins except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

A “subsequence” refers to a sequence of nucleic acids or amino acids that comprise a part of a longer sequence of nucleic acids or amino acids (e.g., protein) respectively.

“Synthetic” refers to a nucleotide sequence comprising structural characters that are not present in the natural sequence. For example, an artificial sequence that resembles more closely the G+C content and the normal codon distribution of dicot and/or monocot genes is said to be synthetic.

Substrate: a substrate is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

Target gene: A “target gene” is any gene in an insect cell. For example, a target gene is a gene of known function or is a gene whose function is unknown, but whose total or partial nucleotide sequence is known. Alternatively, the function of a target gene and its nucleotide sequence are both unknown. A target gene is a native gene of the insect cell or is a heterologous gene that had previously been introduced into the insect cell or a parent cell of said insect cell, for example by genetic transformation. A heterologous target gene is stably integrated in the genome of the insect cell or is present in the insect cell as an extrachromosomal molecule, e.g. as an autonomously replicating extrachromosomal molecule.

Transformation: a process for introducing heterologous DNA into a cell, tissue, or insect Transformed cells, tissues, or insects are understood to encompass not only the end product of a transformation process, but also transgenic progeny thereof.

“Transformed,” “transgenic,” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

Viability: “viability” as used herein refers to a fitness parameter of an insect. Insects are assayed for their homozygous performance of Drosophila development, indicating which proteins are indispensable to maintain life in Drosophila.

### DETAILED DESCRIPTION OF THE INVENTION

I. Identification Of Essential Drosophila melanogaster Nucleotide Sequences Using Transposable Element Insertion Mutagenesis

As shown in Table 2 and the examples below, the identification of novel nucleotide sequences, as well as the essentiality of the nucleotide sequences for normal insect viability, have been demonstrated in Drosophila using P-element transposable insertion mutagenesis. Having established the essentiality of the function of the encoded proteins in Drosophila and having identified the nucleotide sequences encoding these essential proteins, the inventors thereby provide an important and sought-after tool for new insecticide development.

A lethal phenotype caused by insertion of a P-element indicates that the affected nucleotide sequence codes for an essential protein in the insect. The characterization of the insertion site using flanking sequence DNA is needed to associate an individual lethal line with specific nucleotide sequences. Genomic DNA adjacent to the 5′ and/or 3′ end of the P-element from the insertion line is generated using inverse PCR.

TABLE 2
Method of validation of nucleic acid sequences as essential

| SEQ ID | |
|---|---|
| NO | validation method |
| 14 | dsRNA and p-element disruption |
| 16 | p-element disruption |
| 18 | p-element disruption |
| 20 | p-element disruption |
| 22 | p-element disruption |
| 24 | p-element disruption |
| 26 | p-element disruption |
| 28 | p-element disruption |
| 30 | dsRNA |
| 32 | p-element disruption |
| 34 | p-element disruption |
| 36 | p-element disruption |
| 38 | p-element disruption |
| 40 | p-element disruption |
| 42 | dsRNA |
| 44 | p-element disruption |
| 46 | p-element disruption |
| 48 | p-element disruption |
| 50 | p-element disruption |
| 52 | DsRNA |
| 54 | p-element disruption |
| 56 | p-element disruption |
| 58 | p-element disruption |
| 60 | p-element disruption |
| 62 | p-element disruption |
| 64 | p-element disruption |
| 66 | p-element disruption |
| 68 | DsRNA |
| 70 | DsRNA |
| 72 | DsRNA |
| 74 | p-element disruption |
| 76 | p-element disruption |
| 78 | p-element disruption |
| 80 | p-element disruption |
| 82 | p-element disruption |
| 84 | p-element disruption |
| 86 | DsRNA |
| 88 | p-element disruption |
| 90 | p-element disruption |
| 92 | p-element disruption |
| 94 | p-element disruption |
| 96 | p-element disruption |
| 98 | p-element disruption |
| 100 | p-element disruption |
| 102 | p-element disruption |
| 104 | p-element disruption |
| 106 | dsRNA and p-element disruption |
| 108 | p-element disruption |
| 110 | DsRNA |
| 112 | p-element disruption |
| 114 | DsRNA |
| 116 | p-element disruption |
| 118 | p-element disruption |
| 120 | p-element disruption |
| 122 | p-element disruption |
| 124 | p-element disruption |
| 126 | p-element disruption |
| 128 | p-element disruption |
| 130 | p-element disruption |
| 132 | p-element disruption |
| 134 | p-element disruption |
| 136 | p-element disruption |
| 138 | p-element disruption |
| 140 | p-element disruption |
| 142 | p-element disruption |
| 144 | p-element disruption |
| 146 | p-element disruption |
| 148 | p-element disruption |
| 150 | p-element disruption |
| 152 | p-element disruption |
| 154 | p-element disruption |
| 156 | p-element disruption |
| 158 | p-element disruption |
| 160 | DsRNA |
| 162 | p-element disruption |
| 164 | p-element disruption |
| 166 | p-element disruption |
| 168 | p-element disruption |
| 170 | p-element disruption |
| 172 | p-element disruption |
| 174 | p-element disruption |
| 176 | p-element disruption |
| 178 | p-element disruption |
| 180 | p-element disruption |
| 182 | p-element disruption |
| 184 | p-element disruption |
| 186 | p-element disruption |
| 188 | p-element disruption |
| 190 | p-element disruption |
| 192 | p-element disruption |
| 194 | DsRNA |
| 196 | p-element disruption |
| 198 | p-element disruption |
| 200 | p-element disruption |
| 202 | p-element disruption |
| 204 | DsRNA |
| 206 | p-element disruption |
| 208 | p-element disruption |
| 210 | p-element disruption |
| 212 | p-element disruption |
| 214 | DsRNA |
| 216 | p-element disruption |
| 218 | p-element disruption |
| 220 | p-element disruption |
| 222 | DsRNA |
| 224 | p-element disruption |
| 226 | p-element disruption |
| 227 | p-element disruption |
| 228 | p-element disruption |
| 230 | p-element disruption |
| 232 | p-element disruption |
| 234 | p-element disruption |
| 236 | p-element disruption |
| 238 | p-element disruption |
| 240 | p-element disruption |
| 242 | p-element disruption |
| 244 | DsRNA |
| 246 | p-element disruption |
| 248 | p-element disruption |
| 250 | p-element disruption |
| 252 | p-element disruption |
| 254 | p-element disruption |
| 256 | p-element disruption |
| 258 | p-element disruption |
| 260 | p-element disruption |
| 262 | p-element disruption |
| 264 | p-element disruption |
| 266 | p-element disruption |
| 268 | p-element disruption |
| 270 | p-element disruption |
| 272 | dsRNA and p-element disruption |
| 274 | p-element disruption |
| 276 | p-element disruption |
| 278 | p-element disruption |
| 280 | p-element disruption |
| 282 | p-element disruption |
| 284 | p-element disruption |
| 286 | DsRNA |
| 288 | DsRNA |
| 290 | p-element disruption |
| 292 | p-element disruption |
| 294 | p-element disruption |
| 296 | DsRNA |
| 298 | DsRNA |
| 300 | p-element disruption |
| 302 | p-element disruption |
| 304 | p-element disruption |
| 306 | p-element disruption |
| 308 | p-element disruption |
| 310 | p-element disruption |
| 312 | p-element disruption |
| 314 | p-element disruption |
| 316 | p-element disruption |
| 318 | p-element disruption |
| 320 | p-element disruption |
| 322 | p-element disruption |
| 324 | p-element disruption |
| 326 | p-element disruption |
| 328 | p-element disruption |
| 330 | p-element disruption |
| 332 | p-element disruption |
| 334 | p-element disruption |
| 336 | p-element disruption |
| 338 | p-element disruption |
| 340 | p-element disruption |
| 342 | dsRNA and p-element disruption |
| 344 | DsRNA |
| 346 | p-element disruption |
| 348 | dsRNA and p-element disruption |
| 350 | DsRNA |
| 352 | dsRNA and p-element disruption |
| 354 | p-element disruption |
| 356 | p-element disruption |
| 358 | dsRNA and p-element disruption |
| 360 | p-element disruption |
| 362 | p-element disruption |
| 364 | p-element disruption |
| 366 | p-element disruption |
| 368 | DsRNA |
| 370 | p-element disruption |
| 372 | p-element disruption |
| 374 | p-element disruption |
| 376 | p-element disruption |
| 378 | p-element disruption |
| 380 | p-element disruption |

I. Determining the Complete Coding Sequences of the Essential Drosophila Nucleotide Sequences

The essential Drosophila nucleotide sequences are identified by isolating nucleotide sequences flanking the P-element insertion and aligning that sequence with genomic Drosophila sequence obtained from the Celera Drosophila database. The protein prediction for each genomic region is obtained by use of an exon algorithm program such as GeneMark. All exon algorithm programs currently used for prediction of proteins are susceptible to inaccuracies, including incomplete predictions of coding sequences, missing alternative splice variants, combining of nearby exons of adjacent genes, and mistranslation at intron-exon borders. The prediction of a complete coding sequence can be confirmed by several methods including polymerase chain reaction (PCR) amplification using the 5′ and 3′ sequence to verify the message, reverse transcription PCR (rtPCR) using an oligonucleotide internal sequence to identify the 5′ and/or 3′ end, and screening of cDNA libraries from insect tissues with probes made from a particular sequence to isolate a true full-length clone. To confirm that the message size is accurate, a Northern blot can be hybridized with a probe from the nucleotide sequence. In addition, matches to the Drosophila EST database helps to confirm existence of message and gives information about the temporal and spatial pattern of expression. Mutation-causing P elements are known to preferentially cluster in the 5′ region of affected genes (Spradling et al, Proc. Natl. Acad. Sci. USA 92: 10824-10830 (1995)), a tendency that increases the chance of recovering overlaps between short flanking sequences and 5′ ESTs. The present invention therefore provides a number of essential nucleotide sequences as well as the amino acid sequences encoded thereby. cDNA clone sequences are set forth in even numbered SEQ ID NOs:14-380. The corresponding encoded amino acid sequences are set forth in odd numbered SEQ ID NOs:15-381.

The isolated gene sequences disclosed herein may be manipulated according to standard genetic engineering techniques to suit any desired purpose. For example, an entire Drosophila gene sequence or portions thereof may be used as a probe capable of specifically hybridizing to coding sequences and messenger RNAs. To achieve specific hybridization under a variety of conditions, such probes include, e.g. sequences that are unique among insect nucleotide sequences for a particular protein of interest and are at least 10 nucleotides in length, preferably at least 20 nucleotides in length, and most preferably at least 50 nucleotides in length. Such probes are used to amplify and analyze related nucleotide sequences from a chosen organism via PCR. This technique is useful to isolate additional insect nucleotide sequences from a desired organism or as a diagnostic assay to determine the presence of particular nucleotide sequences in an organism. This technique also is used to detect the presence of altered nucleotide sequences associated with a particular condition of interest such as insecticide tolerance, poor health, etc.

Gene-specific hybridization probes also are used to quantify levels of a particular gene mRNA in an insect using standard techniques such as Northern blot analysis. This technique is useful as a diagnostic assay to detect altered levels of gene expression that are associated with particular conditions such as enhanced tolerance to insecticides that target a particular gene.

I.A. Identification of Essential Drosophila melannogaster Nucleotide Sequences Using RNAi

RNA-mediated interference (RNAi) is a recently discovered method to determine gene function in a number of organisms, wherein double-stranded RNA (dsRNA) directs gene-specific, post-transcriptional silencing. See, e.g., Kuwabara & Olson (2000) Parasitol Today 16(8):347-349; Bass (2000) Cell 101(3):235-238; Hunter (2000) Curr Biol 10(4):R137-140; Bosher & Labouesse (2000) Nat Cell Biol 2(2):E31-36; Sharp (1999) Genes Dev 13(2):139-141. The double-stranded RNA molecule can be synthesized in vitro and then introduced into the organism by injection or other methods. Alternatively, a heritable transgene exhibiting dyad symmetry can provide a transcript that folds as a hairpin structure. Methods for examining gene functions using dsRNAi in Drosophila are disclosed in Example 4a and further in Kennerdell & Carthew (2000) Nat Biotech 18(8):896-898; Lam & Thummel (2000) Curr Biol 10(16):957-963; Misquitta & Paterson (1999) Proc Natl Acad Sci USA 96 (4):1451-1456. The present invention describes RNA-mediated interference of sequences listed in Table 2 and Table 6. Double-stranded RNA complementary to each sequence was synthesized in vitro and injected into early Drosophila embryos, as described in Example 4a. Development of injected embryos was assessed by scoring: (a) morphological criteria using a light microscope (Campos-Ortega & Hartenstein (1985) The Embryonic Development of Drosophila melanogaster, Springer-Verlag, Berlin), (b) embryo hatching to become a larvae, (c) puparium formation, and (d) eclosion of the pupae as an adult fly, as indicated in Table 6 herein below. Buffer-injected embryos were injected and monitored in parallel as a control. The percentage of embryos injected with dsRNA that survive to the adult stage is depicted in set forth in Table 6.

Essential genes were identified as those resulting in a percent viable adults below 38% when disrupted by RNAi. This threshold was determined by comparison to multiple buffer-injected controls.

II. Recombinant Production of Protein and Uses Thereof

For recombinant production of a protein of the invention in a host organism, a nucleotide sequence encoding the protein is inserted into an expression cassette designed for the chosen host and introduced into the host where it is recombinantly produced. The choice of the specific regulatory sequences such as promoter, signal sequence, 5′ and 3′ untranslated sequence, and enhancer appropriate for the chosen host is within the level of the skill of the routineer in the art. The resultant molecule, containing the individual elements linking in the proper reading frame, is inserted into a vector capable of being transformed into the host cell. Suitable expression vectors and methods for recombinant production of proteins are well known for host organisms such as E. coli, yeast, and insect cells (see, e.g., Lucknow and Summers, Bio/Technol. 6:47 (1988)). Additional suitable expression vectors are baculovirus expression vectors, e.g., those derived from the genome of Autographica californica nuclear polyhedrosis virus (AcMNPV). A preferred baculovirus/insect system is PVL1392(3) used to transfect Spodoptera frugiperda SF9 cells (ATCC) in the presence of linear Autographica californica baculovirus DNA (Phramingen, San Diego, Calif.). The resulting virus is used to infect HighFive Tricoplusia ni cells (Invitrogen, La Jolla, Calif.).

Recombinantly produced proteins are isolated and purified using a variety of standard techniques. The actual techniques used vary depending upon the host organism used, whether the protein is designed for secretion, and other such factors. Such techniques are well known to the skilled artisan (see, e.g. chapter 16 of Ausubel, F. et al., “Current Protocols in Molecular Biology”, pub. by John Wiley & Sons, Inc. (1994).

IV. Assays for Characterizing the Proteins

Recombinantly produced proteins are useful for a variety of purposes. For example, they can be used in in vitro assays to screen known insecticidal chemicals whose target has not been identified to determine if they inhibit protein activity. Such in vitro assays may also be used as more general screens to identity chemicals that inhibit such protein activity and that are therefore novel insecticide candidates. Recombinantly produced proteins may also be used to elucidate the complex structure of these molecules and to further characterize their association with known inhibitors in order to rationally design new inhibitory insecticides. Alternatively, the recombinant protein can be used to isolate antibodies or peptides that modulate the activity and are useful in transgenic solutions.

V. In Vivo Inhibitor Assay: Discovery of Small Molecule Ligands that Interact with Proteins of Unknown Function.

Having identified a protein as a potential insecticide target based on its essentiality for insect viability, a next step is to develop an assay that allows screening large numbers of chemicals to determine which ones interact with the protein. Although it is straightforward to develop assays for proteins of known function, developing assays with proteins of unknown functions can be more difficult.

To address this issue, novel technologies are used that can detect interactions between a protein and a ligand without knowing the biological function of the protein. A short description of three methods is presented, including fluorescence correlation spectroscopy, surface-enhanced laser desorption/ionization, and biacore technologies. In addition to those descibed here, there are additional methods that are currently being developed that are also amenable to automated, large-scale screening.

Fluorescence Correlation Spectroscopy (FCS) theory was developed in 1972 but it is only in recent years that the technology to perform FCS became available (Madge et al. (1972) Phys. Rev. Lett., 29: 705-708; Maiti et al. (1997) Proc. Natl. Acad. Sci. USA, 94: 11753-11757). FCS measures the average diffusion rate of a fluorescent molecule within a small sample volume. The sample size can be as low as 10³ fluorescent molecules and the sample volume as low as the cytoplasm of a single bacterium. The diffusion rate is a function of the mass of the molecule and decreases as the mass increases. FCS can therefore be applied to protein-ligand interaction analysis by measuring the change in mass and therefore in diffusion rate of a molecule upon binding. In a typical experiment, the target to be analyzed is expressed as a recombinant protein with a sequence tag, such as a poly-histidine sequence, inserted at the N- or C-terminus. The expression takes place in E. coli, yeast or insect cells. The protein is purified by chromatography. For example, the poly-histidine tag can be used to bind the expressed protein to a metal chelate column such as Ni2+ chelated on iminodiacetic acid agarose. The protein is then labeled with a fluorescent tag such as carboxytetramethylrhodamine or BODIPY® (Molecular Probes, Eugene, Oreg.). The protein is then exposed in solution to the potential ligand, and its diffusion rate is determined by FCS using instrumentation available from Carl Zeiss, Inc. (Thornwood, N.Y.). Ligand binding is determined by changes in the diffusion rate of the protein.

Surface-Enhanced Laser Desorption/Ionization (SELDI) was invented by Hutchens and Yip during the late 1980's (Hutchens and Yip (1993) Rapid Commun. Mass Spectrom. 7: 576-580). When coupled to a time-of-flight mass spectrometer (TOF), SELDI provides means to rapidly analyze molecules retained on a chip. It can be applied to ligand-protein interaction analysis by covalently binding the target protein on the chip and analyze by MS the small molecules that bind to this protein (Worrall et al. (1998) Anal. Biochem. 70: 750-756). In a typical experiment, the target to be analyzed is expressed as described for FCS. The purified protein is then used in the assay without further preparation. It is bound to the SELDI chip either by utilizing the poly-histidine tag or by other interaction such as ion exchange or hydrophobic interaction. The chip thus prepared is then exposed to the potential ligand via, for example, a delivery system able to pipet the ligands in a sequential manner (autosampler). The chip is then submitted to washes of increasing stringency, for example a series of washes with buffer solutions containing an increasing ionic strength. After each wash, the bound material is analyzed by submitting the chip to SELDI-TOF. Ligands that specifically bind the target will be identified by the stringency of the wash needed to elute them.

Biacore relies on changes in the refractive index at the surface layer upon binding of a ligand to a protein immobilized on the layer. In this system, a collection of small ligands is injected sequentially in a 2-5 microlitre cell with the immobilized protein. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein (Liedberg et al. (1983) Sensors Actuators 4: 299-304; Malmquist (1993) Nature 361: 186-187). In a typical experiment, the target to be analyzed is expressed as described for FCS. The purified protein is then used in the assay without further preparation. It is bound to the Biacore chip either by utilizing the poly-histidine tag or by other interaction such as ion exchange or hydrophobic interaction. The chip thus prepared is then exposed to the potential ligand via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics on rate and off rate allows the discrimination between non-specific and specific interaction.

The compounds that are active in the methods disclosed herein may be used to combat agricultural pests such as aphids, locusts, spider mites, and boll weavils as well as such insect pests which attack stored grains and against immature stages of insects living on plant tissue. The compounds are also useful as a nematodicide for the control of agriculturally important soil nematodes and plant parasites.

VI. Production of Peptides

Phage particles displaying diverse peptide libraries permits rapid library construction, affinity selection, amplification and selection of ligands directed against an essential protein (H. B. Lowman, Annu. Rev. Biophys. Biomol. Struct. 26, 401-424 (1997)). Structural analysis of these selectants can provide new information about ligand-target molecule interactions and then in the process also provide a novel molecule that can enable the development of new insecticides based upon these peptides as leads.

The invention will be further described by reference to the following detailed examples. These examples are provided for purposes of illustration only, and are not intended to be limiting unless otherwise specified.

### EXAMPLES

Standard recombinant DNA and molecular cloning techniques used here are well known in the art and are described by Sambrook, et al., Molecular Cloning, eds., Cold Spring Harbor Laboratory Press, Cold Spring Harbor, N.Y. (1989) and by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987). Well known Drosophila molecular genetics techniques can be found, for example, in Robert, D. B., Drosophila, A Practical Approach (IRL Press, Washington, D.C., 1986).

### Example 1

### Identification of Lethal Lines

Essential nucleotide sequences are identified through the isolation of lethal mutants defective in development The genetic scheme for mobilization of P-lacW is as performed in Deak et. al, Genetics 147: 1697-1722 (1997). Additional lethal lines are identified and disclosed in Braun, A., B. Lemaitre, et al., Genetics 147: 623-634 (1997); Galloni, M. and B. A. Edgar, Development 126: 2365-2375 (1999); Gateff, E., Int. J. Dev. Biol. 38(4): 565-590 (1994); Mechler, B. M. J. Biosci., Bangalore 19(5): 537-556 (1994); Roch, F., F. Serras, et al., Mol. Gen. Genet. 257: 103-112 (1998); Russell, M. A., L. Ostafichuk, et al., Genome 41: 7-13 (1998); and in Torok, T., G. Tick et al. Genetics 135: 71-80 (1993), Schaefer et al., Aug. 8, 1999 Personal communication to FlyBase. Furthermore, the BDGP gene disruption project of single P-element insertions reveals lethal lines mutating 25% of vital Drosophila genes Spradling, A. C., D. Stern, et al., Genetics 153: 135-177 (1999).

Males carrying the transposase source P(Δ2-3) are crossed en masse to yellow white females homozygous for a P-lacW insertion on the X chromosome. Males carrying the PlacW insertion on the X and Δ2-3 on the third chromosome are collected from this cross. The F0 “jumpstart” males are crossed in groups of 10-15 to 20-25 females of w spl; Sb/TM3, Ser genetype. Male F1 progeny with pigmented eyes indicate that the P-lacW has jumped to an autosome. An average of 10-15 males from each F0 cross lacking Δ2-3 are crossed individually to y w, DTS4/TM3, Sb Ser females, that all third chromosomal insertions result in balanced F2 stocks. Insertions on other autosomes yield white-eyed flies in the F2 generation and are eliminated. The balanced third chromosome insertions are tested for lethality in the next generation by placing four to six pairs of y w; P-lacW/TM3, Sb Ser flies in a vial and examining their progeny for the presence of homozygous P-lacW flies. To analyze the lethal phase, the TM3, Sb Ser balancer is replaced by the TM6C, TB Sb chromosome. In such a genetic background, homozygous mutants can be identified by their wild-type body-length. An average of 10-15 pairs of flies are placed in vials supplemented with yeast paste, and the eggs are collected from each line for 1 day. The development of 50-100 progeny is monitored, and the presence of homozygotes are recorded in all developmental stages. Lethal phase is assigned to a developmental stage in which homozygote animals last appear. Lethal lines are identified and maintained.

TABLE 3
P-element location

| | | Inverse | |
|---|---|---|---|
| seq ID | p-element line | PCR | df cross |
| 14 | l(1)G0335 | 516M3h-f09 | Df(2L)Dwee[wo5] |
| 16 | l(3)064301 | 979H5h-b01 | Previously verified |
| 18 | l(3)092416 | 1022H5h- | Previously verified |
| | | c03 | |
| 20 | l(1)G0384 | 449M3h-b09 | Df(1)JC70/Dp(1; Y)dx[+]5, y[+]/C(1)M5 |
| 22 | l(1)G0449 | 267M3h-d07 | Df(1)JC70/Dp(1; Y)dx[+]5, y[+]/C(1)M5 |
| 24 | l(3)s126215 | 1082H5h- | GN50(63E; 64B) |
| | | f05 | |
| 26 | l(1)G0435 | 661m3h | C(1; Y)1, Df(1)g, y[1] f[1] B[1]/C(1)A, y[1]/Dp(1; f)LJ9, y[+] g[+] na[+] Ste[+] |
| 28 | l(3)079101 | 798H5h-e01 | df 084D04-06; 085B06 |
| 32 | l(3)s147104 | 1108H5h- | 6-7(82D; 82F)by62(85D; 85F) |
| | | h06 | |
| 34 | l(3)047418 | 957H5h-a05 | Previously verified |
| 36 | l(1)G0425 | 619M5h-b- | Dp(1; Y)619, y[+] B[S]/w[1] otd[9]/C(1)DX, y[1] w[1] f[1] |
| | | e10 | |
| 38 | l(3)122404 | 1079H5h- | Previously verified |
| | | f02 | |
| 40 | l(1)G0105 | 360H5hA | Df(1)svr, N[spl-1] ras[2] fw[1]/Dp(1; Y)y[2]67g19.1/C(1)DX, y[1] f[1] |
| 44 | l(3)057809 | 971M5h-e06 | Previously Verified |
| 46 | l(1)G0127 | 373M3h-f03 | Previously Verified |
| 48 | l(1)G0469 | 629H3h-f | C(1; Y)1, Df(1)g, y[1] f[1] B[1]/C(1)A, y[1]/Dp(1; f)LJ9, y[+] g[+] na[+] Ste[+] |
| 50 | l(3)S070103 | 788M5h-h03 | 091F01-02; 092D03-06 BL#3012 |
| 54 | l(3)S104104 | 1057M5h- | Previously Verified |
| | | g08 | |
| 56 | l(3)s090609 | 1017H5h- | emc5(61C; 62A) |
| | | a03 | |
| 58 | l(3)093909 | 1026H5h- | Previously Verified |
| | | a11 | |
| 60 | l(1)G0095 | 354M3h-e10 | Df(1)GE202/Y; Dp(1; 2)sn[+]72d/Dp(?; 2)bw[D], bw[D] |
| 62 | l(1)G0031 | 577M3h-h06 | BL3219 C(1; Y)1, Df(1)g, y[1] f[1] B[1]/C(1)A, y[1]/Dp(1; f)LJ9, y[+] g[+] |
| | | | na[+] Ste[+] |
| 64 | l(1)G0354 | 524M3h-g04 | BL1319 Tp(1; 2)w-ec, ec[64d] cm[1] ct[6] sn[3]/C(1)DX, y[1] w[1] f[1] |
| 66 | l(1)G0062 | 333H5h-b02 | Df(1)R20, y[1?]/C(1)DX, y[1] w[1] f[1]/Dp(1; Y)y[+]mal[+] |
| 74 | l(2)k00237 | AQ034169 | BL3219 C(1; Y)1, Df(1)g, y[1] f[1] B[1]/C(1)A, y[1]/Dp(1; f)LJ9, y[+] g[+] |
| | | | na[+] Ste[+] |
| 76 | l(1)G0181 | 492H3h-f | BL936 Df(1)64c18, g[1] sd[1]/Dp(1; 2; Y)w[+]/C(1)DX, y[1] w[1] f[1] |
| 78 | l(3)078514 | 797H5h-d12 | def. 087D01-02; 088E05-06 |
| 80 | l(3)s112110 | 1069H5h- | ry506(88B; 88D) |
| | | e04 | |
| 82 | l(3)024120 | 930H5h-e06 | Previously verified |
| 84 | l(1)G0150 | 442M3h-b02 | Df(1)R20, y[1?]/C(1)DX, y[1] w[1] f[1]/Dp(1; Y)y[+]mal[+] |
| 88 | l(3)054211 | 968H5h-a09 | Previously verified |
| 90 | l(1)G0399 | 659m3h | BL 901Df(1)svr, N[spl-1] ras[2] fw[1]/Dp(1; Y)y[2]67g19.1/C(1)DX, y[1] f[1] |
| 92 | l(1)G0399 | 659m3h | BL 901Df(1)svr, N[spl-1] ras[2] fw[1]/Dp(1; Y)y[2]67g19.1/C(1)DX, y[1] f[1] |
| 94 | l(3)S104002 | 1061H5h- | W4(75B; 75C)by62(85D; 85F) |
| | | d08 | |
| 96 | l(3)S133705 | 1092M5h- | Previously verified |
| | | f09 | |
| 98 | l(3)041706 | 949H5h-g10 | Previously verified |
| 100 | l(1)G0251 | 392M3h-f11 | Df(1)64c18, g[1] sd[1]/Dp(1; 2; Y)w[+]/C(1)DX, y[1] w[1] f[1] |
| 102 | l(3)100409 | 1050H5h- | crb87-5(95F; 96A) |
| | | c09 | |
| 104 | l(1)G0491 | 643M5h-b- | BL3219 C(1; Y)1, Df(1)g, y[1] f[1] B[1]/C(1)A, y[1]/Dp(1; f)LJ9, y[+] g[+] |
| | | g11 | na[+] Ste[+] |
| 108 | l(1)G0306 | 603m3h | BL1879 Df(1)GE202/Y; Dp(1; 2)sn[+]72d/Dp(?; 2)bw[D], bw[D] |
| 112 | l(1)G0344 | 609H5hA | BL3219 C(1; Y)1, Df(1)g, y[1] f[1] B[1]/C(1)A, y[1]/Dp(1; f)LJ9, y[+] g[+] |
| | | | na[+] Ste[+] |
| 116 | l(3)s083705 | 1006H5h- | 2-2(81F; 82F) |
| | | h07 | |
| 118 | l(1)G0044 | 319M3h-c02 | Df(1)svr, N[spl-1] ras[2] fw[1]/Dp(1; Y)y[2]67g19.1/C(1)DX, y[1] f[1] |
| 120 | l(1)G0012 | 300M5h-b- | Df(1)svr, N[spl-1] ras[2] fw[1]/Dp(1; Y)y[2]67g19.1/C(1)DX, y[1] f[1] |
| | | e08 | |
| 122 | l(1)G0012 | 300M5h-b- | Df(1)svr, N[spl-1] ras[2] fw[1]/Dp(1; Y)y[2]67g19.1/C(1)DX, y[1] f[1] |
| | | e08 | |
| 124 | l(1)G0431 | 566H3h-f | BL 901 Df(1)svr, N[spl-1] ras[2] fw[1]/Dp(1; Y)y[2]67g19.1/C(1)DX, y[1] f[1] |
| 126 | l(1)G0130 | 376H3h-f- | Df(1)svr, N[spl-1] ras[2] fw[1]/Dp(1; Y)y[2]67g19.1/C(1)DX, y[1] f[1] |
| | | e10 | |
| 128 | l(1)G0010 | 576M3h-c07 | BL5279 Df(1)JC70/Dp(1; Y)dx[+]5, y[+]/C(1)M5 |
| 130 | l(3)s118602 | 1076H5h- | ZP1(66A; 66C)G28(66B; 66C)ry506(88B; 88D)red1(88B; 88D) |
| | | e11 | |
| 132 | l(1)G0285 | 508H3h-f- | BL3033 Df(1)R20, y[1?]/C(1)DX, y[1] w[1] f[1]/Dp(1; Y)y[+]mal[+] |
| | | e03 | |
| 134 | l(3)s137212 | 1094H5h- | GN50(63E; 64B) |
| | | g05 | |
| 136 | P{GawB}c338 | F49 (13m3h | |
| 138 | l(1)G0334 | 515M3h-g09 | BL5279 Df(1)JC70/Dp(1; Y)dx[+]5, y[+]/C(1)M5 |
| 140 | l(1)G0464 | 627M3h-d | BL5292 (008C-D; 009B + 001A01; 001B02) |
| 142 | l(3)099013 | 1044H5h- | Previously Verified |
| | | c04 | |
| 144 | l(3)144912 | 1103H5h- | Previously verified |
| | | h01 | |
| 146 | l(1)G0345 | 471M3h-d03 | BL5279 Df(1)JC70/Dp(1; Y)dx[+]5, y[+]/C(1)M5 |
| 148 | l(1)G0453 | 663M3h-d03 | BL5292 y[1] nej[Q7] v[1] f[1]/Dp(1; Y)FF1, y[+]/C(1)DX, y[1] w[1] f[1] |
| 150 | l(1)G038 | 616H5hB | BL 929 Df(1)v-L15, y[1]/C(1)DX, y[1] w[1] f[1]; Dp(1; 2)v[+]75d/+ |
| 152 | l(1)G0492 | 666M3h-d06 | Previously verified |
| 154 | l(1)G0052 | 325M5h-b- | Df(1)v-N48, f[*]/Dp(1; Y)y[+]v[+]#3/C(1)DX, y[1] f[1] |
| | | f01 | |
| 156 | l(1)G0269 | 653M5h-b | BL3033 Df(1)R20, y[1?]/C(1)DX, y[1] w[1] f[1]/Dp(1; Y)y[+]mal[+] |
| 158 | l(1)G0241 | 422H3h-f- | Dp(1; Y)BSC1, y[+]/w[67c23] P{lacW]l(1)G0060[G0060]/C(1)RM, y[1] v[1] |
| | | d02 | |
| 162 | l(1)G0141 | 277M5h-b- | Dp(1; Y)BSC1, y[+]/w[67c23] P{lacW]l(1)G0060[G0060]/C(1)RM, y[1] v[1] |
| | | b08 | |
| 164 | l(1)G0250 | 468H5h-e02 | BL5292 y[1] nej[Q7] v[1] f[1]/Dp(1; Y)FF1, y[+]/C(1)DX, y[1] w[1] f[1] |
| 166 | l(3)sS030003 | 943H5h-e09 | M-Kx1(86C; 87B)T-61(86E; 87A)T32(86E; 87C) |
| 168 | l(1)G0428 | 456M3h-c04 | BL1538 Df(1)os[UE69]/C(1)DX, y[1] f[1]/Dp(1; Y)W39, y[+] ! = fcl[+]Y |
| 170 | l(3)072603 | 996H5h-h02 | previously verified |
| 172 | l(3)S094310 | 1029H5h- | previously verified |
| | | c08 | |
| 174 | l(1)G0220 | 467M3h-d02 | M19 BL1527 Df(1)svr, N[spl-1] ras[2] fw[1]/Dp(1; Y)y[2]67g19.1/C(1)DX, |
| | | | y[1] f[1] |
| 176 | l(3)090417 | 811H5h-e11 | def. 087D01-02; 088E05-06 |
| 178 | l(3)s2172 | AQ034107 | gasfilling screen |
| 180 | l(1)G0025 | 310M3h-d09 | Df(1)JC70/Dp(1; Y)dx[+]5, y[+]/C(1)M5 |
| 182 | l(1)G0076 | 343M34-d11 | Previously verified |
| 184 | l(1)G0151 | 482M3h-g04 | BL1527 Df(1)svr, N[spl-1] ras[2] fw[1]/Dp(1; Y)y[2]67g19.1/C(1)DX, y[1] f[1] |
| 186 | l(3)S069605 | 990M5h-f06 | previously verified |
| 188 | l(1)G0221 | 434H3h-f- | Df(1)19, f[1]/C(1)RM, y[1] shi[1] f[1]; Dp(1; Y)shi[+]3, y[+] |
| | | f02 | |
| 190 | l(1)G0075 | 342M3h-d12 | Df(1)v-N48, f[*]/DP(1; Y)y[+]#3/C(1)DX, y[1] f[1] |
| 192 | l(3)s002001 | 886H5h-c09 | R-G5(62A; 62D)R-G7(62B; 62F) |
| 196 | l(1)G0046 | 321M3h-c04 | Df(1)64c18, g[1] sd[1]/Dp(1; 2; Y)w[+]/C(1)DX, y[1] w[1] f[1] |
| 198 | l(1)G0020 | 303M5h-b- | Dp(1; Y)619, y[+] B[S]/w[1] otd[9]/C(1)DX, y[1] w[1]f[1] |
| | | f06 | |
| 200 | l(3)s095214 | 1032H5h- | faf-BP(100D; 100F) |
| | | b05 | |
| 202 | l(1)G0481 | 275H5bB | Dp(1; Y)619, y[+] B[S]/w[1] otd[9]/C(1)DX, y[1] w[1] f[1] |
| 206 | l(3)s119608 | 1077H5h- | B81(99C; 100F) |
| | | e12 | |
| 208 | l(1)G0172 | 650H3h-f- | BL5292 y[1] nej[Q7] v[1] f[1]/DP(1; Y)FF1, y[+]/C(1)DX, y[1] w[1] f[1] |
| | | c12 | |
| 210 | l(1)G0429 | 564M3h-b11 | BL5459 C(1; Y)6, y[1] w[*] P{white-un4}BE1305 mew[023]/C(1)RM, Y[1] |
| | | | pn[1] v[1]; Dp(1; f)y[+] |
| 212 | l(3)005028 | 892H5h-a04 | Previously verified |
| 216 | l(1)G0343 | 520M5h-b | BL5594 Df(1)dhd81, w[1118]/C(1)DX, y[1] f[1]; Dp(1; 2)4FRDup/+ |
| 218 | l(1)G0343 | 520M5h-b | BL5594 Df(1)dhd81, w[1118]/C(1)DX, y[1] f[1]; Dp(1; 2)4FRDup/+ |
| 220 | l(1)G0174 | 463M3h-c10 | Df(1)dhd81, w[1118]/C(1)DX, y[1] f[1]; Dp(1; 2)4FRDup/+ |
| 224 | l(1)G0132 | 377H3h-f- | Df(1)svr, N[spl-1] ras[2] fw[1]/DP(1; Y)y[2]67g19.1/C(1)DX, y[1] f[1] |
| | | f10 | |
| 226 | l(1)G0144 | 387M3h-f06 | Df(1)64c18, g[1] sd[1]/Dp(1; 2; Y)w[+]/C(1)DX, y[1] w[1] f[1] |
| 228 | l(1)G0144 | 387M3h-f06 | Df(1)64c18, g[1] sd[1]/Dp(1; 2; Y)w[+]/C(1)DX, y[1] w[1] f[1] |
| 230 | l(1)G0312 | 291M5h-b- | Df(1)64c18, g[1] sd[1]/Dp(1; 2; Y)w[+]/C(1)DX, y[1] w[1] f[1] |
| | | g08 | |
| 232 | l(3)S044402 | 954M5h-b06 | Previously Verified |
| 234 | l(1)G0375 | 534M5h-b- | BL936 Df(1)64c18, g[1] sd[1]/Dp(1; 2; Y)w[+]/C(1)DX, y[1] w[1] f[1] |
| | | h03 | |
| 236 | l(1)G0159 | 486M3h-d09 | BL5279 Df(1)JC70/Dp(1; Y)dx[+]5, y[+]/C(1)M5 |
| 238 | l(1)G0227 | 651H3h-f | BL5279 Df(1)JC70/Dp(1; Y)dx[+]5, y[+]/C(1)M5 |
| 240 | l(1)G0212 | 433M3h-a06 | Df(1)19, f[1]/C(1)RM, y[1] shi[1] f[1]; Dp(1; Y)shi[+]3, y[+] |
| 242 | l(1)G0296 | 383H5hA | Df(1)svr, N[spl-1] ras[2] fw[1]/Dp(1; Y)y[2]67g19.1/C(1)DX, y[1]0 f[1] |
| 244 | l(3)j2B9 | AQ026304 | gasfilling screen |
| 248 | l(1)G0007 | 298M3h-a08 | Previously verified |
| 250 | l(3)070006 | 991H5h-b08 | Previously verified |
| 252 | l(1)G0423 | 454M3h-c02 | Df(1)svr, N[spl-1] ras[2] fw[1]/Dp(1; Y)y[2]67g19.1/C(1)DX, y[1] f[1] |
| 254 | l(1)G0361 | 527H3h-f | BL556 Dp(1; Y)BSC1, y[+]/w[67c23] P{lacW]l(1)G0060[G0060]/C(1)RM, |
| | | | y[1] v[1] |
| 256 | l(1)G0290 | 285H5hA | Df(1)JC70/Dp(1; Y)dx[+]5, y[+]/C(1)M5 |
| 258 | l(1)G0436 | 570M3h-c03 | BL 929 Df(1)v-L15, y[1]/C(1)DX, y[1] w[1] f[1]; Dp(1; 2)v[+]75d/+ |
| 260 | l(1)G0111 | 362M5hA | Dp(1; Y)BSC1, y[+]/w[67c23] P{lacW]1(1)G0060[G0060]/C(1)RM, y[1] v[1] |
| 262 | l(1)G0183 | 264H3h-f- | Df(1)svr, N[spl-1] ras[2] fw[1]/Dp(1; Y)y[2]67g19.1/C(1)DX, y[1] f[1] |
| | | e07 | |
| 264 | l(3)S100209 | 1049H5h- | Previously verified |
| | | d08 | |
| 266 | l(3)S100209 | 1049H5h- | Previously verified |
| | | d08 | |
| 268 | l(1)G0438 | 572M3h-c05 | BL5270 Df(1)19, f[1]/C(1)RM, y[1] shi[1] f[1]; Dp(1; Y)shi[+]3, y[+] |
| 270 | l(1)G0116 | 366M5h-b- | Df(1)19, f[1]/C(1)RM, y[1] shi[1] f[1]; Dp(1; Y) shi[+]3, y[+] |
| | | f09 | |
| 272 | l(3)S025007 | 934M5h-g05 | Previously verified |
| 274 | l(1)G0419 | 561M3h-b09 | BL 929 Df(1)v-L15, y[1]/C(1)DX, y[1] w[1] f[1]; Dp(1; 2)v[+]75d/+ |
| 276 | l(3)S008418 | 900H5h-a05 | Previously verified |
| 278 | l(3)141110 | 1098H5h- | Previously verified |
| | | g08 | |
| 280 | l(3)S148011 | 1110H5h- | P115(89B; 89E)C4(89E; 90A) |
| | | g08 | |
| 282 | l(3)S023204 | 923M5h-f05 | Previously verified |
| 284 | l(3)S096404 | 1037H5h- | Previously verified |
| | | a08 | |
| 286 | l(3)145511 | 1104H5h- | Previously verified |
| | | h02 | |
| 292 | l(3)S110013 | 1066H5h- | Previously verified |
| | | h08 | |
| 294 | l(3)010605 | 904H5h-d11 | Previously verified |
| 296 | l(3)100604 | 1051H5h- | Previously verified |
| | | c10 | |
| 302 | l(3)001604 | 883H5h-c06 | Previously verified |
| 304 | l(1)G0358 | 526M3h-g06 | BL1538 Df(1)os[UE69]/C(1)DX, y[1] f[1]/Dp(1; Y)W39, y[+] ! = fcl[+]Y |
| 306 | l(3)067006 | 984H5h-g07 | Previously Verified |
| 308 | l(1)G0070 | 338M3h-d08 | Df(1)os[UE69]/C(1)DX, y[1] f[1]/Dp(1; Y)W39, y[+] ! = fcl[+]Y |
| 310 | l(3)02240 | G00700 | Df(3L)AC1 |
| 312 | l(3)088205 | 1013H5h- | Previously Verified |
| | | c01 | |
| 314 | l(3)S042228 | 951H5h-f01 | vin2(67F; 68D)vin5(68A; 69A) |
| 316 | l(3)S050407 | 964H5h-a07 | M-Kx1(86C; 87B)T-61(86E; 87A)T32(86E; 87C) |
| 318 | l(3)011046 | 908H5h-d09 | Previously verified |
| 320 | l(3)S094204 | 1028H5h- | ea(88E; 89A) |
| | | b01 | |
| 322 | l(3)001917 | 738H5h-a03 | def. 089E01-F04; 091B01-B02 |
| 324 | l(3)131602 | 858H5h-h10 | def. 089E01-F04; 091B01-B02 |
| 326 | l(1)G0451 | 624M3h-a10 | BL 901 Df(1)svr, N[spl-1] ras[2] fw[1]/Dp(1; Y)y[2]67g19.1/C(1)DX, y[1] f[1] |
| 328 | l(3)S022231 | 920H5h-g04 | Previously verified |
| 330 | l(3)S085401 | 225M3d | Df(3L-Xs-533/TM6B Sb[1]Ser[1] (76B4-77B) |
| 332 | l(3)075515 | 794H5h-d09 | def. 076B04; 077B |
| 334 | l(3)131602 | 858H5h-h10 | def. 089E01-F04; 091B01-B02 |
| 336 | l(3)058302 | 972H5h-a11 | Previously verified |
| 338 | l(3)058302 | 972H5h-a11 | Previously verified |
| 340 | l(3)S005916 | 895H5h-d01 | lxd6(67F; 68D)P14(90C; 91A) |
| 342 | l(3)025616 | 752H5h-b02 | def. 087D01-02; 088E05-06 |
| 348 | l(3)S089302 | 1014H5h- | AC1(67A; 67D) |
| | | a01 | |
| 354 | l(2)06444 | AQ025653 | In(2R)vg[W] |
| 356 | l(3)026115 | 938H5h-e07 | Previousyl verified |
| 358 | l(1)G0461 | 626M3h-a12 | BL5279 Df(1)JC70/Dp(1; Y)dx[+]5, y[+]/C(1)M5 |
| 360 | l(2)04329 | G00564 | Df(2R)vg135 Df(2R)CX1 |
| 362 | l(3)113105 | 1070H5h- | Previously verified |
| | | e05 | |
| 364 | l(1)G0213 | 495M5h-b | BL1537 Dp(1; Y)W73, y[31d] B[1], f[+], B[S]/C(1)DX, y[1] f[1]/y[1] |
| | | | baz[EH171] |
| 366 | l(3)003606 | 888H5h-d06 | Previously verified |
| 368 | l(3)S005042 | 893H5h-c01 | eN19(93B; 94)eR1(93B; 93D) |
| 372 | l(3)S075101 | 1002H5h- | pXT103(85A; 85C) |
| | | h04 | |
| 374 | l(1)G0455 | 269H5h-a01 | BL5678 duplication |
| 376 | l(1)G0260 | 432M3h-a05 | Df(1)19, f[1]/C(1)RM, y[1] shi[1] f[1]; Dp(1; Y)shi[+]3, y[+] |
| 378 | l(3)S086909 | 806H5h-b04 | 087D01-02; 088E05-06 BL1534 |
| 380 | l(1)G0272 | 435H3h-f- | M26 BL5270 Df(1)19, f[1]/C(1)RM, y[1] shi[1] f[1]; Dp(1; Y)shi[+]3, y[+] |
| | | g02 | |

### Example 2

### Sequence Determination

Inverse PCR: To determine the flanking sequence of the lethal lines, the “Inverse PCR and Cycle Sequencing Protocol for Recovery of Sequences Flanking PZ, PlacW; and PEP elements” of E. Jay Rehm, Berkeley Drosophila Genome Project on the world wide web at fruitfly.org/methods/ is used with slight modifications. These modifications include the following: genomic DNA is obtained from 10 flies, rather than 30 flies, with adjustments for final concentrations; all DNA precipitations are performed using glycogen; for some reactions, the digest volume is used in the appropriate ligations; the number of cycles in PCR reactions was increased to 40; Pry1 and Pry2 were used to sequence the PEP line flanking sequences.

Genomic DNA isolation: Flies are collected and frozen at −20° C. until ready for use. Genomic DNA is prepared by grinding flies in 200 μl Buffer A with a disposable grinder 30× (Buffer A is composed of 100 mM Tris-Cl, pH7.5, 100 mM EDTA, 100 mM NaCl, 0.5% SDS). Add 200 μl additional Buffer A; grind another 15×. Keep on ice until finished. Incubate at 65° C. for 30 minutes. Vortex to mix. Add 800 μl freshly made LiCl/KAc Solution (LiCl/Kac Solution is comprised of 1 part 5 M KAc and 2.5 parts 6 M LiCl). Vortex. Incubate −20° C. for 20 minutes. Spin at maximum speed at room temperature 15+ minutes. Transfer 1 ml supernatant to a clean tube avoiding floating debris. Add 600 μl room temperature isopropanol to supernatant. Mix well by tipping. Add 0.5 μl glycogen. Vortex. Incubate at room temperature for 5 minutes. Spin 15 minutes at room temperature, maximum speed. Aspirate away the supernatant Wash 2× with 500 μl 70% room temperature ethanol; vortex between washes. Spin for 10 minutes at room temperature, maximum speed. Aspirate away supernatant. Dry in a speed vacuum for 10 minutes. Resuspend in 50 μl TE+0.1 mg/ml RNAse A {for 1 ml TE/RNAse A Solution, add 990 μl TE+10 μl RNAse A (10 mg/ml)). Check 5 μl on 0.8% gel.

Digest Genomic DNA (Sau3A I, HinP1I, or Msp I—done separately): Set up digests in 96 well tray. Per reaction, add 10 μl genomic DNA, 5 μl 10× Buffer, 2 μl 0.1 mg/ml RNAase A stock, 30.5 μl dH₂O, 10 units of enzyme (8 units for Sau 3AI), 0.5 μl of 100×BSA (for Sau 3AI only). Incubate at 37° C. for 2.5 hours. Check on 0.8% gel before heat-inactivating at 65° C. for 20 minutes.

Ligate P Element and Flanking DNA: Set-up ligation tube with 400 μl of ligation mixture then add 30-50 μl of the digest: Per reaction, add 30 μl of digested genomic DNA, 43 μl of 10× ligation buffer (NEB), 375 μl of dH₂O, and 2 μl of ligase (2 Weiss units). Incubate overnight at 4° C. Total reaction volume is adjusted as appropriate.

Precipitate Ligated DNA: To ligation tube, add 40 μl 3M NaAc pH5.2+1 ml 100% room temperature ethanol+1 μl glycogen. Mix by tipping. Incubate −20° C. for 15+ minutes. Spin 15 minutes, 4° C. Aspirate away supernatant Wash with 500 μl room temperature 70% ethanol. Vortex. Spin room at temperature for 10 minutes. Aspirate away supernatant. Dry in speed vacuum for 10 minutes. Resuspend in 50μl TE. Vortex to mix. Transfer to 96 well plate.

PCR: Set up PCR reactions in 96 well plates (Applied Biosystems). Set up PCR reactions with primers appropriate for the type of P element and the end of the element from which genomic sequence is to be recovered.

Primers for PCR: (type of P element 5′ or 3′ end forward primer reverse primer annealing temperature):

| | |
|---|---|
| PZ P-element5′ endPlac4Plac1 | 60° |
| PZ P-element3′ endPry4Pry1 | 55° |
| PZ P-element3′ endPry2Pry1 | 60° |
| PlacW P-element5′ endPlac4Plac1 | 60° |
| PlacW P-element3′ endPry4Plw3-1 | 55° |
| PlacW P-element3′ endPry2Pry1 | 60° |
| PEP P-element5′ endPwht1Plac1 | 60° |
| PEP P-element3′ endPry4Pry1 | 55° |
| PEP P-element3′ endPry2Pry1 | 60° |

The Pry2/Pry1 combination has a higher annealing temperature than the Pry4/Pry1 and Pry4/Plw3-1 combinations, but the resulting PCR products do not allow sequencing directly off the 3′ end of the P-element. The latter primer combinations are therefore used in all initial experiments; the Pry2/Pry1 combination can be used in those cases where strong and unique bands do not result.

Per reaction: 10 μl of ligated genomic DNA, 1 μl of 10 mM dNT mix, 1 μl of 10 μM forward primer stock, 1 μl of 10 μl reverse primer stock, 5 μl of 10× Qiagen Taq buffer, 31.5 μl of dH₂O, 0.5 μl of Qiagen Taq.

Cycles: 1×95° C. for 5 minutes; 40×(95° C. for 30 seconds; 60° C. (high temp) or 55° C. (low temp) for 30 seconds; 68° C. for 2 minutes); 1×72° C. for 10 minutes; hold at 4° C.; run 10 μl on 1.5% gel to check. Rearray positive wells to 96 well plate for sequencing clean-up. The primer sets for PCR are as shown in the table below:

TABLE 4
PCR Primers

| Digest, End, Temperature | Forward PCR Primer | Reverse PCR Primer |
|---|---|---|
| H5h | Plac4 | Plac1 |
| H3h | Pry2 | Pry1 |
| H3l | Pry4 | Plw3-1 |
| M5h | Plac4 | Plac1 |
| M3h | Pry2 | Pry1 |
| M3l | Pry4 | Plw3-1 |
| S5h | Plac4 | Plac1 |
| S3h | Pry2 | Pry1 |
| S3l | Pry4 | Plw3-1 |

PCR Primer Sequences (5′ to 3′):

| | |
|---|---|
| Plac4 (27) | |
| -act gtg cgt tag gtc ctg ttc att gtt | SEQ ID NO:1 |
| Plac1 (24) | |
| -cac cca agg ctc tgc tcc cac aat | SEQ ID NO:2 |
| Pry4 (23) | |
| -caa tca tat cgc tgt ctc act ca | SEQ ID NO:3 |
| Pry1 (26) | |
| -cct tag cat gtc cgt ggg gtt tga at | SEQ ID NO:4 |
| Pry2 (28) | |
| -ctt gcc gac ggg acc acc tta tgt tat t | SEQ ID NO:5 |
| Plw3-1 (19) | |
| -tgt cgg cgt cat caa ctc c | SEQ ID NO:6 |
| Pwht1 (19) | |
| -gta acg cta atc act ccg aac agg tca ca | SEQ ID NO:7 |

Enzymatic Clean-Up for Sequencing: To 40 μl PCR reaction, add 4 μl of enzyme mix. Incubate at 37° C. for 1 hour. Inactivate at 70° C. for 10 minutes. (Enzyme Mix consists of 2.5 U/μl Exonuclease I (Amersham E700732), 0.5 U/μl Shrimp Alkaline Phosphatase (Amersham E70183), 1× Amplitaq PCR buffer, add dH₂O to final volume.)

### Example 3

### Sequence Analysis

Sequence of the flanking sequence generated by inverse PCR is performed on an ABI 3700 sequencer (Perkin Elmer) using BIG DYE sequencing reaction.

Primer sets for sequencing are as shown in the table below:

TABLE 5
PCR Primers for Flanking Sequences

| Digest, End, Temperature | Forward Primer | Reverse Primer |
|---|---|---|
| H5h | Splac2 | Sp1 |
| H3h | Pry2 | Sp5 |
| H3l | Spep1 | Sp5 |
| M5h | Splac2 | Sp1 |
| M3h | Pry2 | Sp5 |
| M3l | Spep1 | Sp5 |
| S5h | Splac2 | Sp1 |
| S3h | Pry2 | Sp6 |
| S3l | Spep1 | Sp6 |

The following primer sets are designed to sequence both ends of PCR products recovered from PlacW and PZ strains:

Splac2 and Sp1—for use with the Plac4/Plac1 5′ PCR primer combination with either PZ or PlacW P-elements; allows sequencing of both ends of the PCR fragment.

Spep1 and Sp3—for use with the Pry4/Pry1 3′ PCR primer combination with PZ P-elements; allows sequencing of both ends of the PCR fragment.

Spep1 and Sp6—for use with the Pry4/Plw3-1 3′ PCR primer combination with PlacW P-elements where Sau3a digestion is performed; allows sequencing of both ends of the PCR fragment.

Spep1 and Sp5—for use with the Pry4/Plw3-1 3′ PCR primer combination where HinP1 digestion is performed; allows sequencing of both ends of the PCR fragment.

Pry1 and Pry2—for use with the Pry1/Pry2 3′ PCR primer combination; allows sequencing of both ends of the PCR fragment.

The PCR products recovered from PEP strains are sequenced with the following primers: Sp1—for use with the Pwht1/Plac1 5′ PCR primer combination with the PEP element; Spep1—for use with the Pry4/Pry1 3′ PCR primer combination with the PEP element; Pry1 and Pry2 for use with the Pry1/Pry2 3′ PCR primer combination with the PEP element.

Primer Sequences (5′ to 3′):

| | |
|---|---|
| Splac2 (25) | |
| -gaa ttc act ggc cgt cgt ttt aca a | SEQ ID NO:8 |
| Sp1 (22) | |
| -aca caa cct ttc ctc tca aca a | SEQ ID NO:9 |
| Sp3 (24) | |
| -gag tac gca aag ctt taa cta tgt | SEQ ID NO:10 |
| Sp6 (23) | |
| -tga cca cat cca aac atc ctc tt | SEQ ID NO:11 |
| Sp5 (25) | |
| -gca tca caa aaa tcg acg ctc aag t | SEQ ID NO:12 |
| Spep1 (19) | |
| -gac act cag aat act att c | SEQ ID NO:13 |

Melting temperatures of sequencing primers:
Splac2—60.1° C.
Sp1—50.6° C.
Sp3—49.3° C.
Sp6—54.9° C.
Sp5—60.3° C.
Spep1—44.8° C.

### Example 4

### Secondary Confirmation of Lethality

The lethality of the chromosome carrying the P-element insertion is demonstrated genetically as described in Example 1. The essential Drosophila nucleotide sequences are identified by isolating nucleotide sequences flanking the P-element insertion and aligning those sequences with genomic Drosophila sequence obtained from the Celera Drosophila database. However, in some instances, a second site mutation exists on the chromosome that is responsible for the lethality. In other instances, the location of the flanking sequence is such that determination of which gene(s) are affected by the P-element insertion is rendered difficult or impossible. Thus, to provide secondary confirmation that the gene indicated is essential, there are many methods that one skilled in the art can use, e.g., rescue of the lethality using transformation technology, perturbation of the gene in a targeted manner, or failure to complement a deficiency.

To provide secondary confirmation, lethal lines are crossed to a line containing a deficiency. This creates a hemizygous condition in that particular region and reveals the recessive phenotype of the P-element. Complementation with deficiencies that unequivocally remove the P-element insertion site is taken as proof that the P-element does not cause the associated phenotype. Failure to complement indicates that the strain is verified. This method is as performed in Spradling, A. C., D. Stern, et al., Genetics 153: 135-177 (1999). If the insert is present on the X chromosome, which is present in two copies in females but only one copy in males, then the recessive phenotype of the P-element insert is revealed by this hemizygous condition in males. A rescue cross is performed to a stock containing a duplication spanning the region of the insert on the X chromosome on one of the autosomes. If the males survive then the presence of an essential gene disrupted by the P-element but rescued by the duplication is confirmed. While lines with secondary mutations closely linked to the P insertion might be erroneously verified by these procedures, further molecular and genetic analyses suggest that the frequency of such errors is small. RNA interference, described in Fire, A., S. Xu, et al., Nature 391, 806-811 (1998) and Kennerdell, J. R. and Carthew, R. W., Cell 95, 1017-1026 (1998), is used as a method to target a gene of interest and demonstrate that the perturbation of the identified gene produces a lethal phenotype.

### Example 4

### Double-Stranded RNA Interference

Preparation of dsRNA for Injection. Sequences to be expressed as dsRNA were cloned into Bluescript KS(+) (Stratagene of La Jolla, Calif.), linearized with the appropriate restriction enzymes, and transcribed in vitro with the Ambion T3 and T7 Megascript kits following the manufacturer's instructions (Ambion Inc. of Austin, Tex.). Transcripts were annealed in injection buffer (0.1 mM NaPO₄ pH 7.8, 5mM KCl) after heating to 85° C. and cooling to room temperature over a 1- to 24 hr period. All annealed transcripts were analyzed on agarose gels with DNA markers to confirm the size of the annealed RNA and quantitated as described previously (Fire et al. (1998) Nature 391(6669):806-811). Injected RNA was not gel-purified. Injection of 0.1 nl of a 0.1- to 1.0-mg/ml solution of a 1-kb dsRNA corresponds to roughly 10⁷ molecules/injection.

Injection of Drosophila melanogaster Embryos. Fly cages were set up using 2- to 4day flies. Agar-grape juice plates were replaced every hour to synchronize the egg collection for 1-2 days. The eggs were collected over a 30-to 60-min period for subsequent injection. The eggs were washed into a nylon mesh basket with tap water. The chorion was removed by brief soaking in a dilute bleach solution. Eggs were positioned on a glass slide such that each egg was in a same orientation. Double-stranded RNA was injected into middle of each egg using an Eppendorf transjector (Eppendorf Scientific, Inc. of Westbury, N.Y.). Following injection, slides were stored in a moist chamber to prevent dessication of the embryos. Embryos were monitored for development and transferred as first intar larvae to vials containing Drosophila medium. Methods for rearing Drosophila staging and common genetic techniques can be found, for example, in Roberts (1986) Drosophila melanogaster, A Practical Approach, IRL Press, Washington, D.C.; Ashburner (1989a) Drosophila: A Laboratory Handbook, Cold Spring Harbor Laboratory Press, New York, N.Y.; Ashburner (1989b) Drosophila: A Laboratory Manual, Cold Spring Harbor Laboratory Press, New York, N.Y.; Goldstein & Fyrberg, eds (1994) in Methods in Cell Biology. Vol. 44, Academic Press, San Diego, Calif.

The data in Table 6 demonstrates the lethal effect of disrupting the production of protein from the message of the specified gene through RNAi. Based on data from postitve and negative controls, a reduction in survival (% viable adults from developed eggs) below 38% represents a significant lethal effect. Many genes show a complete loss of survivability (with 0% viable). Others show a range of phenotypic penetrance, which is most likely due to the variability of the RNAi technique, but are still considered lethals because they are significantly below controls.

TABLE 6
Data for dsRNA Interference

| | | | # eggs | | | | % viable |
|---|---|---|---|---|---|---|---|
| | | | showing | | | | adults from |
| | Inventor's | # eggs | morphological | # hatched | | | developed |
| seq ID | reference | Injected | development | larvae | # pupae | # adults | eggs |
| | none, buffer only | 941 | 806 | 580 | 500 | 433 | 53.72 |
| 14 | GIN00231, CT28483 | 163 | 148 | 107 | 28 | 26 | 17.57 |
| 30 | GIN00961, CT31117 | 472 | 386 | 170 | 8 | 1 | 0.26 |
| 42 | GIN01243, CT36241 | 107 | 99 | 81 | 9 | 7 | 7.07 |
| 52 | GIN01682, CT1465 | 140 | 127 | 87 | 23 | 15 | 11.81 |
| 68 | GIN01885, CT13424 | 170 | 154 | 78 | 17 | 8 | 5.19 |
| 70 | GIN01896, CT14932 | 164 | 140 | 78 | 44 | 38 | 27.14 |
| 72 | GIN01977, CT23511 | 79 | 70 | 18 | 17 | 15 | 21.43 |
| 86 | GIN02340, CT28931 | 190 | 159 | 0 | 0 | 0 | 0.00 |
| 106 | GIN03775, CT33819 | 172 | 148 | 16 | 0 | 0 | 0.00 |
| 110 | GIN03797, CT33841 | 136 | 127 | 12 | 0 | 0 | 0.00 |
| 114 | GIN04053, CT3509 | 168 | 145 | 106 | 1 | 1 | 0.69 |
| 160 | GIN05757, CT4810 | 159 | 144 | 109 | 37 | 32 | 22.22 |
| 194 | GIN07111, CT6007 | 159 | 140 | 94 | 0 | 0 | 0.00 |
| 204 | GIN07278, CT6738 | 174 | 166 | 7 | 3 | 1 | 0.60 |
| 214 | GIN07446, CT9021 | 125 | 119 | 1 | 0 | 0 | 0.00 |
| 222 | GIN07609, CT6171 | 372 | 316 | 119 | 0 | 0 | 0.00 |
| 246 | GIN08205, CT12517 | 717 | 569 | 433 | 26 | 25 | 4.39 |
| 274 | GIN08858, CT14874 | 177 | 161 | 13 | 3 | 3 | 1.86 |
| 288 | GIN09788, CT17938 | 100 | 83 | 71 | 5 | 2 | 2.41 |
| 290 | GIN09819, CT17971 | 181 | 142 | 107 | 7 | 1 | 0.70 |
| 298 | GIN10338, CT19788 | 170 | 137 | 88 | 5 | 1 | 0.73 |
| 300 | GIN10364, CT19850 | 58 | 55 | 47 | 14 | 6 | 10.91 |
| 344 | GIN11831, CT24122 | 103 | 87 | 0 | 0 | 0 | 0.00 |
| 346 | GIN11918, CT24346 | 469 | 408 | 301 | 257 | 88 | 21.57 |
| 350 | GIN11993, CT24437 | 145 | 130 | 93 | 0 | 0 | 0.00 |
| 352 | GIN12074, CT18257 | 104 | 93 | 80 | 3 | 3 | 3.23 |
| 354 | GIN12174, CT24731 | 168 | 145 | 122 | 1 | 1 | 0.69 |
| 360 | GIN12437, CT25274 | 473 | 424 | 334 | 237 | 63 | 14.86 |
| 370 | GIN13270, CT27543 | 101 | 92 | 78 | 2 | 2 | 2.17 |

### Example 5

### Isolation of Full Length cDNA

A cDNA screen is performed using a Drosophila melanogaster cDNA library probed with a portion of each nucleotide sequence disclosed in the Sequence Listing. Positive colonies are selected, a subset sequenced, and a clone corresponding to the full-length cDNA is recovered. Alternatively, primers from the predicted 5′ and 3′ end are used in polymerase chain reaction with either a Drosophila cDNA library or first strand cDNAs obtained by reverse transcription of Drosophila mRNAs as template to amplify a fragment representing the full-length clone.

### Example 6

### Expression of Recombinant Protein in Insect Cells

Baculovirus vectors, which are derived from the genome of AcNPV virus, are designed to provide high levels of expression of cDNA in the SF9 line of insect cells (ATCC CRL#1711). Recombinant baculovirus expressing the cDNA of the present invention is produced by the following standard methods (InVitrogen MaxBac Manual): cDNA constructs are ligated into the polyhedrin gene in a variety of baclovirus transfer vectors, including the pAC360 and the BleAc vector (InVitrogen). Recombinant baculoviruses are generated by homologous recombination following co-transfection of the baculovirus transfer vector and linearized AcNPV genomic DNA (Kitts, P. A., Nucleic Acid Res. 18: 5667 (1990)) into SF9 cells. Recombinant pAC360 viruses are identified by the absence of inclusion bodies in infected cells and recombinant pBlueBac viruses are identified on the basis of B-galactosidase expression (Summers, M. D. and Smith, G. E., Texas Agriculture Exp. Station Bulletin No. 1555). Following plaque purification, the Drosophila cDNA expression is measured.

The cDNA encoding the entire open reading frame for the Drosophila cDNA is inserted into the BamHI site of pBlueBacII. Constucts in the positive orientation, which are identified by sequence analysis, are used to transfect SF9 cells in the presence of linear AcNPV wild type DNA. Authentic, active Drosophila cDNA is found in the cytoplasm of infected cells. Active Drosophila cDNA is extracted from infected cells by hypotonic or detergent lysis.

### Example 7

### Expression of Recombinant Protein in E. coli

A cDNA clone of the present invention is subcloned into an appropriate expression vector and transformed into E. coli using the manufacturer's conditions. Specific examples include plasmids such as pBluescript (Stratagene, La Jolla, Calif.), pFLAG (International Biotechnologies, Inc., New Haven, Conn.), and pTrcHis (Invitrogen, La Jolla, Calif.). E. coli is cultured, and expression of the recombinant protein is confirmed. Recombinant protein is then isolated using standard techniques.

### Example 8

### In Vitro Binding Assays

Recombinant protein is obtained, for example according to Example 6 or Example 7. The protein is immobilized on chips appropriate for ligand binding assays. The protein immobilized on the chip is exposed to sample compound in solution according to methods well know in the art. While the sample compound is in contact with the immobilized protein measurements capable of detecting protein-ligand interactions are conducted. Examples of such measurements are SEDLI, biacore and FCS, described above. Compounds found to bind the protein are readily discovered in this fashion and are subjected to further characterization.

The above disclosed embodiments are illustrative. This disclosure of the invention will place one skilled in the art in possession of many variations of the invention. All such obvious and foreseeable variations are intended to be encompassed by the appended claims.

The numerous publications and patents referred to in this document are hereby incorporated by reference, in their entirety.

What is claimed is:

1. A method for identifying a compound that inhibits the activity of a protein essential for Drosophila viability, comprising:
(a) expressing in a recombinant host a DNA molecule comprising
(i) a nucleotide sequence selected from the group consisting of the even numbered SEQ ID NOs:14-380, or
(ii) a nucleotide sequence encoding an amino acid sequence selected from the group consisting of the odd numbered SEQ ID NOs:15-381,
to produce a protein essential for Drosophila viability;
(b) testing compounds suspected of having the ability to inhibit the activity of the protein expressed in (a); and
(c) identifying a compound tested in (b) that inhibits the activity of the protein.

2. A method for killing or inhibiting the growth or viability of an insect, comprising applying to the insect a compound identified according to the method of claim 1.

3. A method for identifying a compound that interacts with a protein essential for Drosophila viability, comprising:
(a) expressing in a recombinant host a DNA molecule comprising
(i) a nucleotide sequence selected from the group consisting of the even numbered SEQ ID NOs:14-380, or
(ii) a nucleotide sequence encoding an amino acid sequence selected from the group consisting of the odd numbered SEQ ID NOs:15-381,
to produce a protein essential for Drosophila viability;
(b) testing compounds suspected of having the ability to interact with the protein expressed in (a); and
(c) identifying a compound tested in (b) that interacts with the protein.

4. A method for killing or inhibiting the growth or viability of an insect, comprising applying to the insect a compound identified according to the method of claim 3.

5. A method for killing or inhibiting the growth or viability of an insect, comprising inhibiting expression in said insect of a protein having at least 60% sequence identity to an amino acid sequence selected from the group consisting of the odd numbered SEQ ID NOs:15-381.

6. The method of claim 5, wherein expression of said protein is inhibited by disruption in said insect of a nucleotide sequence having at least 60% sequence identity to a nucleotide sequence selected from the group consisting of the even numbered SEQ ID NOs:14-380.

7. The method of claim 6, wherein said nucleotide sequence is disrupted by RNA interference.
